(19) United States
(12) Patent Application Publication
(10) Pub. No.: US 2008/0009439 A1
(43) Pub. Date: Jan. 10, 2008

(54) Compositions and Methods of Use for a Fibroblast Growth Factor

(76) Inventors: John P. Alsobrook, Corrales, NM (US); Joseph Eichen, East Haven, CT (US); Denise Lepley, Hartford, CT (US); Charles E. Miller, Abingdon, MD (US); Peter Mezes, Old Lyme, CT (US)

Correspondence Address: Ivor R. Elrifi;Mintz, Levin, Cohn, Ferris, Glovsky and Popeo, One Financial Center, Boston, MA, 02111, US

(21) Appl. No.: 10/578,234

(22) PCT Filed: Nov. 3, 2004
(86) PCT No.: PCT/US04/36478

§ 371 Date: May 14, 2007

Related U.S. Application Data
(63) Continuation of application No. 10/702,126, filed on Nov. 4, 2003, now abandoned.
(60) Provisional application No. 60/423,747, filed on Nov. 5, 2002.
(60) Provisional application No. 60/469,353, filed on May 9, 2003.

(51) Int. Cl. A61K 38/00; A61P 43/00; C07K 14/00; C12N 1/20; C12N 15/00; C12N 15/11; C12P 21/04; C12Q 1/68; G01N 33/53

(52) U.S. Cl. 514/12; 435/252.3; 435/252.33; 435/320.1; 435/6; 435/69.1; 435/7.21; 530/350; 536/22.1

(57) ABSTRACT

The present invention generally relates to compositions and methods of use for a fibroblast growth factor (“FGF”). More particularly, the present invention relates to a nucleotide polymorphism (SNP) of FGF-20, and its fragments, derivatives, variants, homologs, and analogs, as well as their uses in wound healing, and prevention and/or treatment of certain diseases, including but are not limited to, stroke, radiation sickness, mucositis, inflammatory bowel disease, and inflammation (osteoarthritis and rheumatoid arthritis).

### FIELD OF THE INVENTION

The present invention relates to compositions and methods of use for a fibroblast growth factor (“FGF”). More particularly, the present invention relates to a nucleotide polymorphism (SNP) of FGF-20, and its fragments, derivatives, variants, homologs, and analogs, as well as their uses in wound healing, and prevention and/or treatment of certain diseases, including but not limited to, radiation sickness, mucositis, inflammatory bowel disease, arthritis, stroke, and neurodegenerative diseases.

### BACKGROUND OF THE INVENTION

The FGF family consists of more than 20 members, each containing a conserved amino acid core (see, e.g., Powers et al., Endocr. Relat. Cancer, 7(3):65-197 (2000)). FGFs regulate diverse cellular functions such as growth, survival, apoptosis, motility, and differentiation (see, e.g., Szebenyi et al., Int. Rev. Cytol., 185:45-106 (1999)). Members of the FGF family are involved in various physiological and pathological processes during embryogenesis and adult life, including morphogenesis, limb development, tissue repair, inflammation, angiogenesis, and tumor growth and invasion (see, e.g., Powers et al., Endocr. Relat. Cancer, 7(3):165-197 (2000); or Szebenyi et al., Int. Rev. Cytol. 185:45-106 (1999)).

The Fibroblast Growth Factor (FGF) family of proteins, whose prototypic members include acidic FGF (FGF-1) and basic FGF (FGF-2), binds to four related receptor tyrosine kinases. These FGF receptors are expressed on most types of cells in tissue culture. Dimerization of FGF receptor monomers upon ligand binding has been reported to be a requisite for activation of the kinase domains, leading to receptor trans- phosphorylation. FGF receptor-1 (FGFR-1), which shows the broadest expression pattern of the four FGF receptors, contains at least seven tyrosine phosphorylation sites. A number of signal transduction molecules are affected by binding with different affinities to these phosphorylation sites.

Expression of FGFs and their receptors in brains of perinatal and adult mice has been examined. Messenger RNA of all FGF genes, with the exception of FGF-4, is detected in these tissues. FGF-3, FGF-6, FGF-7 and FGF-8 genes demonstrate higher expression in the late embryonic stages than in postnatal stages, suggesting that these members are involved in the late stages of brain development. In contrast, expression of FGF-1 and FGF-5 increased after birth. In particular, FGF-6 expression in perinatal mice has been reported to be restricted to the central nervous system and skeletal muscles, with intense signals in the developing cerebrum in embryos but in cerebellum in 5-day-old neonates. FGF-receptor (FGFR)-4, a cognate receptor for FGF-6, demonstrate similar spatiotemporal expression, suggesting that FGF-6 and FGFR-4 plays significant roles in the maturation of nervous system as a ligand-receptor system. These results strongly suggest that the various FGFs and their receptors are involved in the regulation of a variety of developmental processes of brain, such as proliferation and migration of neuronal progenitor cells, neuronal and glial differentiation, neurite extensions, and synapse formation.

Other members of the FGF protein family include the FGF receptor tyrosine kinase (FGFRTK) family and the FGF receptor heparan sulfate proteoglycan (FGFRHS) family. These members interact to regulate active and specific FGFR signal transduction complexes. These regulatory activities are diversified throughout a broad range of organs and tissues, and in both normal and tumor tissues, in mammals. Regulated alternative messenger RNA (mRNA) splicing and combination of variant subdomains give rise to diversity of FGFRTK monomers. Divalent cations cooperate with the FGFRHS to conformationally restrict FGFRTK trans-phosphorylation, which causes depression of kinase activity and facilitates appropriate activation of the FGFR complex by FGF. For example, it is known that different point mutations in the FGFRTK commonly cause craniofacial and skeletal abnormalities of graded severity by graded increases in FGF-independent activity of total FGFR complexes. Other processes in which FGF family exerts important effects are liver growth and function and prostate tumor progression.

Glia-activating factor (GAF), another FGF family member, is a heparin-binding growth factor that was purified from the culture supernatant of a human glioma cell line (See Mol Cell Biol 13(7): 4251-4259, 1993). GAF shows a spectrum of activity slightly different from those of other known growth factors, and is designated as FGF-9. The human FGF-9 cDNA encodes a protein of 208 amino acids. Sequence similarity to other members of the FGF family was estimated to be around 30%. Two cysteine residues and other consensus sequences found in other family members were also well conserved in the FGF-9 sequence. FGF-9 was found to have no typical signal sequence in its N terminus like those in acidic FGF and basic FGF. Acidic FGF and basic FGF are known not to be secreted from cells in a conventional manner. However, FGF-9 was found to be secreted efficiently from cDNA-transfected COS cells despite its lack of a typical signal sequence. It could be detected exclusively in the culture medium of cells. The secreted protein lacked no amino acid residues at the N terminus with respect to those predicted by the cDNA sequence, except the initiation methionine. The rat FGF-9 cDNA was also cloned, and the structural analysis indicated that the FGF-9 gene is highly conserved.

Through a homology-based genomic mining process, a new member of the FGF family, FGF-20, has been identified. See U.S. patent application Ser. Nos. 09/494,585, filed Jan. 13, 2000, and 09/609,543, filed Jul. 3, 2000, the content of each application is incorporated herein by reference in its entirety.

Citation or discussion of a reference herein shall not be construed as an admission that such is prior art to the present invention.

### SUMMARY OF THE INVENTION

In one aspect, the invention provides an isolated CG53135-12 family nucleic acid that encodes a CG53135-12 family protein, or a fragment, homolog, analog or derivative thereof. The nucleic acid can include, e.g., nucleic acid sequence encoding a protein at least 85% identical to a protein comprising the amino acid sequence of SEQ ID NOs: 2, 6, 8, 11, 13, 15, 17, 19, 21, 23, or 25. Also included in the invention is a vector containing one or more of the nucleic acids described herein, and a cell containing the vectors or nucleic acids described herein.

In general, an CG53135-12-like variant that preserves CG53135-12 function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution.

The present invention is also directed to host cells transformed with a recombinant expression vector comprising any of the nucleic acid molecules described above.

In one aspect, the invention includes a pharmaceutical composition that includes a CG53135-12 family nucleic acid and a pharmaceutically acceptable carrier. In a further aspect, the invention includes a substantially purified CG53135-12 family protein, e.g., any of the CG53135-12 family proteins encoded by a CG53135-12 family nucleic acid, and fragments, homologs, analogs, and derivatives thereof. The invention also includes a pharmaceutical composition that includes a CG53135-12 family protein and a pharmaceutically acceptable carrier.

In a further aspect, the invention provides an antibody that binds specifically to a CG53135-12 family protein. The antibody can be, e.g., a monoclonal or polyclonal antibody, and fragments, homologs, analogs, and derivatives thereof. The invention also includes a pharmaceutical composition including CG53135 antibody and a pharmaceutically acceptable carrier or diluent. The present invention is also directed to isolated antibodies that bind to an epitope on a protein encoded by any of the nucleic acid molecules described above.

The present invention is further directed to kits comprising antibodies that bind to a protein encoded by any of the nucleic acid molecules described above and a negative control antibody.

The invention further provides a method for producing a CG53135-12 family protein. The method includes providing a cell containing a CG53135-12 family nucleic acid, e.g., a vector that includes a CG53135-12 family nucleic acid, and culturing the cell under conditions sufficient to express the CG53135-12 family protein encoded by the nucleic acid. The expressed CG53135-12 family protein is then recovered from the cell. Preferably, the cell produces little or no endogenous CG53135-12 family protein. The cell can be, e.g., a prokaryotic cell or eukaryotic cell.

The present invention provides a method of inducing an immune response in a mammal against a protein encoded by any of the nucleic acid molecules disclosed above by administering to the mammal an amount of the protein sufficient to induce the immune response.

The present invention is also directed to methods of identifying a compound that binds to CG53135-12 family protein by contacting the CG53135-12 family protein with a compound and determining whether the compound binds to the CG53135-12 family protein.

The invention further provides methods of identifying a compound that modulates the activity of a CG53135-12 family protein by contacting CG53135-12 family protein with a compound and determining whether the CG53135-12 family protein activity is modified.

The present invention is also directed to compounds that modulate CG53135-12 family protein activity identified by contacting a CG53135-12 family protein with the compound and determining whether the compound modifies activity of the CG53135-12 family protein, binds to the CG53135-12 family protein, or binds to a nucleic acid molecule encoding a CG53135-12 family protein.

In another aspect, the invention provides a method of diagnosing a tissue proliferation-associated disorder, such as tumors, restenosis, psoriasis, diabetic and post-surgery complications, and arthritis (e.g., osteoarthritis, rheumatoid arthritis), in a subject. The method includes providing a protein sample from the subject and measuring the amount of CG53135-12 family protein in the subject sample. The amount of CG53135-12 family in the subject sample is then compared to the amount of CG53135-12 family protein in a control protein sample. An alteration in the amount of CG53135-12 family protein in the subject protein sample relative to the amount of CG53135-12 family protein in the control protein sample indicates the subject has a tissue proliferation-associated condition. A control sample is preferably taken from a matched individual, i.e., an individual of similar age, sex, or other general condition but who is not suspected of having a tissue proliferation-associated condition. Alteratively, the control sample may be taken from the subject at a time when the subject is not suspected of having a tissue proliferation-associated disorder. In some embodiments, the CG53135-12 family protein is detected using a CG53135-12 FAMILY antibody.

The invention is also directed to methods of inducing an immune response in a mammal against a protein encoded by any of the nucleic acid molecules described above. The method includes administering to the mammal an amount of the protein sufficient to induce the immune response.

In a further aspect, the invention includes a method of diagnosing a tissue proliferation-associated disorder, such as tumors, restenosis, psoriasis, diabetic and post-surgery complications, and arthritis (e.g., osteoarthritis, rheumatoid arthritis), in a subject. The method includes providing a nucleic acid sample, e.g., RNA or DNA, or both, from the subject and measuring the amount of the CG53135-12 family nucleic acid in the subject nucleic acid sample. The amount of CG53135-12 family nucleic acid sample in the subject nucleic acid is then compared to the amount of CG53135-12 family nucleic acid in a control sample. An alteration in the amount of CG53135-12 FAMILY nucleic acid in the sample relative to the amount of CG53135-12 family in the control sample indicates the subject has a tissue proliferation-associated disorder.

In a further aspect, the invention includes a method of diagnosing a tissue proliferation-associated disorder in a subject. The method includes providing a nucleic acid sample from the subject and identifying at least a portion of the nucleotide sequence of a CG53135-12 family nucleic acid in the subject nucleic acid sample. The CG53135-12 family nucleotide sequence of the subject sample is then compared to a CG53135-12 family nucleotide sequence of a control sample. An alteration in the CG53135-12 family nucleotide sequence in the sample relative to the CG53135-12 family nucleotide sequence in said control sample indicates the subject has a tissue proliferation-associated disorder.

In a still further aspect, the invention provides method of treating or preventing or delaying a tissue proliferation-associated disorder, cancer, oral mucositis, radiation sickness, inflammatory bowel disease, ischemic stroke, hemorrhagic stroke, trauma, spinal cord damage, heavy metal or toxin poisoning, neurodegenerative diseases (such as Alzheimer's, Parkinson's Disease, Amyotrophic Lateral Sclerosis, Huntington's Disease) rheumatoid arthritis and osteoarthritis.

The method includes administering to a subject in which such treatment or prevention or delay is desired a CG53135-12 family nucleic acid, a CG53135-12 family protein, or a CG53135-12 family antibody in an amount sufficient to treat, prevent, or delay a tissue proliferation-associated disorder in the subject.

The tissue proliferation-associated disorders diagnosed, treated, prevented or delayed using the CG53135-12 family nucleic acid molecules, proteins or antibodies can involve epithelial cells, e.g., fibroblasts and keratinocytes in the anterior eye after surgery. Other tissue proliferation-associated disorder include, e.g., tumors, restenosis, psoriasis, Dupuytren's contracture, diabetic complications, Kaposi sarcoma, and rheumatoid arthritis.

For further overview of FGF-related techniques U.S. Ser. No. 10/435087 is incorporated herein in toto. Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 (A) This figure depicts the major allelic form FGF20 homolog FGF9, with an Aspartic acid residue at position 203 (SNP CG53135-12 at aa 206 in CG531354-01 (FGF-20)). It forms a single intramolecular bond (dashed line inside the circle) with Arginine 63 (conserved at aa 66 in CG53135-01). (B) This figure depicts the minor allelic form of the homolog FGF9, corresponding to SNP CG53135-12 in CG531355-01. An Asparagine residue has been substituted at position 203 (206 in CG53135-01). It forms 2 additional intramolecular bonds with Histidine 56 (conserved at aa 59 in CG53135-01) (dashed lines inside upper circle), while maintaining the intramolecular bond described in FIG. 18A (dashed line inside lower circle).

FIG. 2. This figure depicts Receptor Binding Specificity of CG53135. NIH 3T3 cells were serum-starved, incubated with the indicated factor (squares=platelet derived growth factor; triangles=FGF-1; circles=CG53135) either alone or together with the indicated soluble FGFR, and DNA synthesis in response to CG53135 was measured in a BrdU incorporation assay. Data points represent the average obtained from triplicate wells, and are represented as the percent BrdU incorporation relative to cells receiving factor alone.

FIG. 3. Chromatogram of CG53135-05 (FGF-20) (black) and CG53135-12 (grey) monitored at 280 nm.

FIG. 4. Deconvoluted mass spectrum of CG53135-05 (FGF-20) (Upper) and CG53135-12 (Lower).

FIG. 5. This figure depicts Proliferation of NIH-3T3 murine fibroblasts in the presence of CG53135-12.

FIG. 6. This figure depicts Proliferation of Balb/MK murine keratinocytes in the presence of CG53135-12.

FIG. 7. This figure depicts Proliferation of Baf3 murine pre-B cell line in the presence of CG53135-12.

FIG. 8. This figure depicts Proliferation of Baf3 murine pre-B cell line transfected with the mouse FGF receptor R1b, in the presence of CG53135-12.

FIG. 9. This figure depicts Proliferation of Baf3 murine pre-B cell line transfected with the mouse FGF receptor R1c, in the presence of CG53135-12.

FIG. 10. Proliferation of Baf3 murine pre-B cell line transfected with the mouse FGF receptor R2b, in the presence of CG53135-12.

FIG. 11. This figure depicts Proliferation of Baf3 murine pre-B cell line transfected with the mouse FGF receptor R2c, in the presence of CG53135-12.

FIG. 12. This figure depicts Proliferation of Baf3 murine pre-B cell line transfected with the mouse FGF receptor R3b, in the presence of CG53135-12.

FIG. 13. This figure depicts Proliferation of Baf3 murine pre-B cell line transfected with the mouse FGF receptor R3c, in the presence of CG53135-12.

FIG. 14. This figure depicts Size Exclusion Chromatography results of CG53135-12.

FIG. 15. Relative binding affinities of FGF-20 (CG53135-05) and CG53135-12.

FIG. 16. (A) Relative binding of FGF-20 (CG53135-05) and CG53135-12 to FGF receptors, 100 nM. (B) Relative binding of FGF-20 (CG53135-05) and CG53135-12 to FGF receptors, 500 nM

### DETAILED DESCRIPTION OF THE INVENTION

The present invention is based, in part, on the discovery of a single nucleotide polymorphism (SNP) in the gene encoding FGF-20 in humans, and the discovery that carriers of this allele have decreased risk for osteoarthritis. This FGF-20 SNP is designated as CG53135-12. The present invention provides the nucleic acids encoding the CG53135-12 protein, the protein encoded by the nucleic acids, and its fragments, derivatives, variants, homologs, and analogs. The present invention also provides methods of use for CG53135-12, as well as for its fragments, derivatives, variants, homologs, and analogs.

For clarity of disclosure, and not by way of limitation, the detailed description of the invention is divided into the following subsections:
(i) CG53135-12
(ii) Preparation of CG53135-12
(iii) Structure Prediction and Functional Analysis of CG53135-12
(iv) Therapeutic and Prophylactic Uses of CG53135-12
(v) Pharmaceutical Compositions
CG53135-12

The present invention provides a single nucleotide polymorphism (SNP) of FGF-20, and its fragments, derivatives, variants, homologs, or analogs. As used herein, the term “CG53135-12” refers to nucleic acid molecules encoding SEQ ID NO:2, or its fragments, derivatives, variants, homologs, or analogs, and the proteins (including peptides and proteins) encoded by such nucleic acid molecules. In one embodiment, the invention includes a variant of CG53135-12 protein, in which some amino acids residues, e.g., no more than 1%, 2%, 3%, 5%, 10% or 15% of the amino acid sequence of CG53135-12, are changed. In another embodiment, the invention includes nucleic acid molecules that can hybridize to a CG53135-12 nucleic acid under stringent hybridization conditions.

As used herein, the term “FGF-20” refers to a protein comprising an amino acid sequence of SEQ ID NO: 4, or a nucleic acid sequence encoding such a protein and/or a complimentary strand thereof.

As used herein, the term “hybridizes under stringent conditions” describes conditions for hybridization and washing under which nucleotide sequences at least 30% (preferably, 35%, 40%, 45%, 50%, 55%, 60%, 65%, 70%, 75%, 80%, 85%, 90%, 95% or 98%) identical to each other typically remain hybridized to each other. Such stringent conditions are known to those skilled in the art and can be found in Current Protocols in Molecular Biology, John Wiley & Sons, N.Y. (1989), 6.3.1-6.3.6. In one, non limiting example, stringent hybridization conditions comprise a salt concentration from about 0.1 M to about 1.0 M sodium ion, a pH from about 7.0 to about 8.3, a temperature is at least about 60° C., and at least one wash in 0.2×SSC, 0.01% BSA. In another non-limiting example, stringent hybridization conditions are hybridization at 6× sodium chloride/sodium citrate (SSC) at about 45° C., followed by one or more washes in 0.1×SSC, 0.2% SDS at about 68° C. In yet another non-limiting example, stringent hybridization conditions are hybridization in 6×SSC at about 45° C., followed by one or more washes in 0.2×SSC, 0.1% SDS at 50-65° C. (i.e., one or more washes at 50° C., 55° C., 60° C. or 65° C.). It is understood that the nucleic acids of the invention do not include nucleic acid molecules that hybridize under these conditions solely to a nucleotide sequence consisting of only A or T nucleotides.

As used herein, the term “isolated” in the context of a protein agent refers to a protein agent that is substantially free of cellular material or contaminating proteins from the cell or tissue source from which it is derived, or substantially free of chemical precursors or other chemicals when chemically synthesized. The language “substantially free of cellular material” includes preparations of a protein agent in which the protein agent is separated from cellular components of the cells from which it is isolated or recombinantly produced. Thus, a protein agent that is substantially free of cellular material includes preparations of a protein agent having less than about 30%, 20%, 10%, or 5% (by dry weight) of host cell proteins (also referred to as a “contaminating proteins”). When the protein agent is recombinantly produced, it is also preferably substantially free of culture medium, i.e., culture medium represents less than about 20%, 10%, or 5% of the volume of the protein agent preparation. When the protein agent is produced by chemical synthesis, it is preferably substantially free of chemical precursors or other chemicals, i.e., it is separated from chemical precursors or other chemicals that are involved in the synthesis of the protein agent. Accordingly, such preparations of a protein agent have less than about 30%, 20%, 10%, 5% (by dry weight) of chemical precursors or compounds other than the protein agent of interest. In a specific embodiment, protein agents disclosed herein are isolated.

As used herein, the term “isolated” in the context of nucleic acid molecules refers to a nucleic acid molecule that is separated from other nucleic acid molecules that are present in the natural source of the nucleic acid molecule. Moreover, an “isolated” nucleic acid molecule, such as a cDNA molecule, can be substantially free of other cellular material or culture medium when produced by recombinant techniques, or substantially free of chemical precursors or other chemicals when chemically synthesized. In a specific embodiment, nucleic acid molecules are isolated.

As used herein, the term “effective amount” refers to the amount of a therapy (e.g., a composition comprising a CG53135 protein) which is sufficient to reduce and/or ameliorate the severity and/or duration of a disease or one or more symptoms thereof, prevent the advancement of a disease, cause regression of a disease, prevent the recurrence, development, or onset of one or more symptoms associated with a disease, or enhance or improve the prophylactic or therapeutic effect(s) of another therapy.

As used herein, the terms “subject” and “subjects” refer to an animal, preferably a mammal, including a non-primate (e.g., a cow, pig, horse, cat, or dog), a primate (e.g., a monkey, chimpanzee, or human), and more preferably a human. In a certain embodiment, the subject is a mammal, preferably a human, who has been exposed to or is going to be exposed to an insult that may induce alimentary mucositis (such as radiation, chemotherapy, or chemical warfare agents). In another embodiment, the subject is a farm animal (e.g., a horse, pig, or cow) or a pet (e.g., a dog or cat) that has been exposed to or is going to be exposed to a similar insult.

Identification of CG53135-12

A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, however, in the case that a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern for example, alteration in temporal expression, physiological response regulation, cell type expression regulation, intensity of expression, stability of transcribed message.

CG53135-12 is a SNP of FGF-20, where the guanine (G) at position 616 of FGF-20 DNA sequence (SEQ ID NO: 3) is replaced with adenine (A). Such replacement results in one amino acid change in the encoded protein: i.e., aspartic acid (Asp) at position 206 of FGF-20 amino acid sequence (SEQ ID NO: 4) is replaced with Asparagine (Asn). Aspartic acid is an acidic, and negative polar amino acid, and asparagines is an uncharged polar amino acid.

CG53135-12 was identified by analyzing sequence assemblies derived from various human samples. cDNA was obtained from various human samples representing multiple tissue types, normal and diseased states, physiological states, and developmental states from different donors. The cDNAs thus derived were then sequenced, and sequence traces were evaluated and edited for corrections if appropriate. The cDNA sequences from all samples were assembled with themselves and with public ESTs using bioinformatics programs. Each assembly can represent a gene and/or its variants such as splice forms and/or single nucleotide polymorphisms and their combinations. SNPs such identified can then be confirmed using one or more validated methods known in the art, e.g., pyrosequencing.

In a study where suitable cases were recruited by sampling 2000 index cases from a knee replacement register, and control subject sampled from the same base population (e.g., matched for genetic background, age range, sex and other demographic factors), it was found that carriers of the minor allele (“A” allele, CG53135-12) are significantly less likely to have osteoarthritis than individuals who are homozygous for the major allele (“G” allele).

CG53135-12 Derivatives, Variants, Homologs, Analogs and Fragments

The present invention also provides derivatives, variants, homologs, analogs and fragments of CG53135-12 (collectively termed “CG53135-12 family”). In some embodiments, a member of the CG53135-12 family is FGF-20. In some embodiments, a member of the CG53135-12 family is not FGF-20. Table 1 shows some of the examples of CG53135-12 variants, homologs and fragments.

TABLE 1
Examples of CG53135-12 Family

| | SEQ ID NO | |
|---|---|---|
| Name | (DNA/Protein) | Brief Description |
| CG53135-12 | 1 and 2 | FGF-20 SNP, aspartic acid at position 206 is changed to |
| | | asparagines (“²⁰⁶D→N”) as compared to FGF-20 (SEQ ID |
| | | NO: 3 and 4) |
| CG53135-12 (02) | 5 and 6 | Codon optimized, amino acids 2-54 (as numbered in SEQ |
| | | ID NO: 2) were removed |
| CG53135-12 (04) | 7 and 8 | Amino acids 20-51 (as numbered in SEQ ID NO: 2) were |
| | | removed, also valine at position 85 is changed to alanine |
| | | (“⁸⁵V→A”) |
| CG53135-12 (05) | 9 and 2 | Codon optimized CG53135-12 |
| CG53135-12 (06) | 10 and 11 | Amino acids 20-51 (as numbered in SEQ ID NO: 2) were |
| | | removed |
| CG53135-12 (13) | 12 and 13 | CG53135-12 minus first 2 amino acids at the N-terminus |
| CG53135-12 (14) | 14 and 15 | CG53135-12 minus first 8 amino acids at the N-terminus |
| CG53135-12 (15) | 16 and 17 | CG53135-12 minus first 11 amino acids at the N-terminus |
| CG53135-12 (16) | 18 and 19 | CG53135-12 minus first 14 amino acids at the N-terminus |
| CG53135-12 (17) | 20 and 21 | CG53135-12 minus first 23 amino acids at the N-terminus |
| CG53135-12 | 22 and 23 | amino acids 63-211 of CG53135-12 (SEQ ID NO: 2) |
| (250059669) | | |
| CG53135-12 | 24 and 11 | amino acids 1-19 and 52-211 of CG53135-12 (SEQ ID |
| (250059596) | | NO: 2) |

In one embodiment, the present invention provides a variant of CG53135-12. In another embodiment, CG53135 refers to a nucleic acid molecule encoding a CG53135-12 protein from other species or the protein encoded thereby, and thus has a nucleotide or amino acid sequence that differs from the human sequence of CG53135-12. Nucleic acid molecules corresponding to natural allelic variants and homologues of the CG53135-12 cDNAs of the invention can be isolated based on their homology to the human CG53135-12 nucleic acids disclosed herein using the human cDNAs, or a portion thereof, as a hybridization probe according to standard hybridization techniques under stringent hybridization conditions.

In another embodiment, the present invention provides a fragment of CG53135-12 protein, including fragments of CG53135-12 variant. An example of a CG53135-12 protein fragment includes, but is not limited to, residues 2-211, 3-211, 9-211, 12-211, 15-211, 24-211, 54-211, or 55-211 of CG53135-12 (SEQ ID NO:2). In one embodiment, the present invention provies a nucleic acid encodes a protein fragment that includes residues 2-211, 3-211, 9-211, 12-211, 15-211, 24-211, 54-211, or 55-211 of SEQ ID NO:2.

The invention also encompasses derivatives and analogs of CG53135-12. The production and use of derivatives and analogs related to CG53135-12 are within the scope of the present invention.

In a specific embodiment, the derivative or analog is functionally active, i.e., capable of exhibiting one or more functional activities associated with a full-length, wild-type CG53135-12. Derivatives or analogs of CG53135-12 can be tested for the desired activity by procedures known in the art, including but not limited to, using appropriate cell lines, animal models, and clinical trials.

In particular, CG53135-12 derivatives can be made via altering CG53135-12 sequences by substitutions, insertions or deletions that provide for functionally equivalent molecules. In one embodiment, such alteration of CG53135-12 sequence is done in a region that is not conserved in the FGF protein family. Due to the degeneracy of nucleotide coding sequences, other DNA sequences which encode substantially the same amino acid sequence as CG53135-12 may be used in the practice of the present invention. These include, but are not limited to, nucleic acid sequences comprising all or portions of CG53135-12 that are altered by the substitution of different codons that encode a functionally equivalent amino acid residue within the sequence, thus producing a silent change. In a preferred embodiment, a wild-type CG53135-12 nucleic acid sequence is codon-optimized to the nucleic acid sequence of SEQ ID NO:8 (CG53135-12 (05)). Likewise, the CG53135-12 derivatives of the invention include, but are not limited to, those containing, as a primary amino acid sequence, all or part of the amino acid sequence of CG53135-12 including altered sequences in which functionally equivalent amino acid residues are substituted for residues within the sequence resulting in a silent change. For example, one or more amino acid residues within the sequence can be substituted by another amino acid of a similar polarity that acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. CG53135-12 derivatives of the invention also include, but are not limited to, those containing, as a primary amino acid sequence, all or part of the amino acid sequence of CG53135-12 including altered sequences in which amino acid residues are substituted for residues with similar chemical properties. In a specific embodiment, 1, 2, 3, 4, or 5 amino acids are substituted.

Derivatives or analogs of CG53135-12 include, but are not limited to, those proteins which are substantially homologous to CG53135-12 or fragments thereof, or whose encoding nucleic acid is capable of hybridizing to the CG53135-12 nucleic acid sequence.

The CG53135-12 derivatives and analogs of the invention can be produced by various methods known in the art. The manipulations that result in their production can occur at the gene or protein level. For example, the cloned CG53135-12 gene sequence can be modified by any of numerous strategies known in the art (e.g., Maniatis, T., 1989, Molecular Cloning, A Laboratory Manual, 2d ed., Cold Spring Harbor Laboratory, Cold Spring Harbor, N.Y.). The sequence can be cleaved at appropriate sites with restriction endonuclease(s), followed by further enzymatic modification if desired, isolated, and ligated in vitro. In the production of the gene encoding a derivative or analog of CG53135-12, care should be taken to ensure that the modified gene remains within the same translational reading frame as CG53135-12 , uninterrupted by translational stop signals, in the gene region where the desired CG53135-12 activity is encoded.

Additionally, the CG53135-12 -encoding nucleic acid sequence can be mutated in vitro or in vivo, to create and/or destroy translation, initiation, and/or termination sequences, or to create variations in coding regions and/or form new restriction endonuclease sites or destroy preexisting ones, to facilitate further in vitro modification. Any technique for mutagenesis known in the art can be used, including but not limited to, in vitro site-directed mutagenesis (Hutchinson, C. et al., 1978, J. Biol. Chem 253:6551), use of TAB.RTM. linkers (Pharmacia), etc.

Manipulations of the CG53135-12 sequence may also be made at the protein level. Included within the scope of the invention are CG53135-12 fragments or other derivatives or analogs which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited to, reagents useful for protection or modification of free NH2— groups, free COOH— groups, OH— groups, side groups of Trp-, Tyr-, Phe-, His-, Arg-, or Lys-; specific chemical cleavage by cyanogen bromide, hydroxylamine, BNPS-Skatole, acid, or alkali hydrolysis; enzymatic cleavage by trypsin, chymotrypsin, papain, V8 protease, NaBH4; acetylation, formylation, oxidation, reduction; metabolic synthesis in the presence of tunicamycin; etc.

In addition, analogs and derivatives of CG53135-12 can be chemically synthesized. For example, a protein corresponding to a portion of CG53135-12 which comprises the desired domain, or which mediates the desired aggregation activity in vitro, or binding to a receptor, can be synthesized by use of a peptide synthesizer. Furthermore, if desired, nonclassical amino acids or chemical amino acid analogs can be introduced as a substitution or addition into the CG53135-12 sequence. Non-classical amino acids include, but are not limited to, the D-isomers of the common amino acids, α-amino isobutyric acid, 4-aminobutyric acid, hydroxyproline, sarcosine, citrulline, cysteic acid, t-butylglycine, t-butylalanine, phenylglycine, cyclohexylalanine, β-alanine, designer amino acids such as β-methyl amino acids, Cα-methyl amino acids, and Nα-methyl amino acids.

In a specific embodiment, the CG53135-12 derivative is a chimeric or fusion protein comprising CG53135-12 or a fragment thereof fused via a peptide bond at its amino- and/or carboxy-terminus to a non-CG53135-12 amino acid sequence. In one embodiment, the non-CG53135-12 amino acid sequence is fused at the amino-terminus of a CG53135-12 member or a fragment thereof. In another embodiment, such a chimeric protein is produced by recombinant expression of a nucleic acid encoding the protein (comprising a CG53135-12-coding sequence joined in-frame to a non-CG53135-12 coding sequence). Such a chimeric product can be custom made by a variety of companies (e.g., Retrogen, Operon, etc.) or made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g., by use of a peptide synthesizer. In a specific embodiment, a chimeric nucleic acid encoding CG53135-12 with a heterologous signal sequence is expressed such that the chimeric protein is expressed and processed by the cell to the mature CG53135-12 protein. The primary sequence of CG53135-12 and non-CG53135-12 gene may also be used to predict tertiary structure of the molecules using computer simulation (Hopp and Woods, 1981, Proc. Natl. Acad. Sci. U.S.A. 78:3824-3828); the chimeric recombinant genes could be designed in light of correlations between tertiary structure and biological function. Likewise, chimeric genes comprising an essential portion of CG53135-12 molecule fused to a heterologous (non-CG53135-12) protein-encoding sequence may be constructed. In a specific embodiment, such chimeric construction can be used to enhance one or more desired properties of CG53135-12, including but not limited to, CG53135-12 stability, solubility, or resistance to proteases. In another embodiment, chimeric construction can be used to target CG53135-12 to a specific site. In yet another embodiment, chimeric construction can be used to identify or purify CG53135-12 family members of the invention, such as a His-tag, a FLAG tag, a green fluorescence protein (GFP), β-galactosidase, a maltose binding protein (MalE), a cellulose binding protein (CenA) or a mannose protein, etc. In one embodiment, a CG53135-12 family member protein is carbamylated.

In some embodiment, a CG53135-12 family member protein can be modified so that it has an extended half-life in vivo using any methods known in the art. For example, inert polymer molecules such as high molecular weight polyethyleneglycol (PEG) can be attached to a CG53135-12 family member protein with or without a multifunctional linker either through site-specific conjugation of the PEG to the N- or C-terminus of the protein or via epsilon-amino groups present on lysine residues. Linear or branched polymer derivatization that results in minimal loss of biological activity will be used. The degree of conjugation can be closely monitored by SDS-PAGE and mass spectrometry to ensure proper conjugation of PEG molecules to the CG53135-12 protein. Unreacted PEG can be separated from CG53135-12-PEG conjugates by size-exclusion or by ion-exchange chromatography. PEG-derivatized conjugates can be tested for in vivo efficacy using methods known to those of skill in the art.

A CG53135-12 family member protein can also be conjugated to, e.g., albumin or Fc fragment of IgG (preferably human IgG) in order to make the protein more stable in vivo or have a longer half life in vivo. The techniques are well known in the art, see e.g., International Publication Nos. WO 93/15199, WO 93/15200, and WO 01/77137; and European Patent No. EP 413, 622, all of which are incorporated herein by reference.

Preparation of CG53135-12

Examples of methods of isolating CG53135-12 protein are described in Section 6, infra, Any techniques known in the art can be used in purifying a CG53135-12 family protein, including but not limited to, separation by precipitation, separation by adsorption (e.g., column chromatography, membrane adsorbents, radial flow columns, batch adsorption, high-performance liquid chromatography, ion exchange chromatography, inorganic adsorbents, hydrophobic adsorbents, immobilized metal affinity chromatography, affinity chromatography), or separation in solution (e.g., gel filtration, electrophoresis, liquid phase partitioning, detergent partitioning, organic solvent extraction, and ultrafiltration). See e.g., Scopes, PROTEIN PURIFICATION, PRINCIPLES AND PRACTICE, 3rd ed., Springer (1994). During the purification, the biological activity of CG53135 may be monitored by one or more in vitro or in vivo assays. The purity of the isolated protein can be assayed by any methods known in the art, such as but not limited to, gel electrophoresis. See Scopes, supra. In some embodiment, the CG53135-12 family proteins employed in a composition of the invention can be in the range of 80 to 100 percent of the total mg protein, or at least 80%, at least 85%, at least 90%, at least 95%, or at least 98% of the total mg protein. In one embodiment, one or more CG53135-12 family proteins employed in a composition of the invention is at least 99% of the total protein. In another embodiment, a CG53135-12 family protein is purified to apparent homogeneity, as assayed, e.g., by sodium dodecyl sulfate polyacrylamide gel electrophoresis.

Methods known in the art can be utilized to recombinantly produce CG53135-12 family member proteins. A nucleic acid sequence encoding a CG53135-12 family member protein can be inserted into an expression vector for propagation and expression in host cells.

An expression construct, as used herein, refers to a nucleic acid sequence encoding a CG53135-12 family protein operably associated with one or more regulatory regions that enable expression of a CG53135-12 family protein in an appropriate host cell. “Operably-associated” refers to an association in which the regulatory regions and the CG53135-12 family member sequence to be expressed are joined and positioned in such a way as to permit transcription, and ultimately, translation.

The regulatory regions necessary for transcription of a CG53135-12 family member can be provided by the expression vector. A translation initiation codon (ATG) may also be provided if a CG53135-12 family member gene sequence lacking its cognate initiation codon is to be expressed. In a compatible host-construct system, cellular transcriptional factors, such as RNA polymerase, will bind to the regulatory regions on the expression construct to effect transcription of the modified CG53135-12 family member sequence in the host organism. The precise nature of the regulatory regions needed for gene expression may vary from host cell to host cell. Generally, a promoter is required which is capable of binding RNA polymerase and promoting the transcription of an operably-associated nucleic acid sequence. Such regulatory regions may include those 5′ non-coding sequences involved with initiation of transcription and translation, such as the TATA box, capping sequence, CAAT sequence, and the like. The non-coding region 3′ to the coding sequence may contain transcriptional termination regulatory sequences, such as terminators and polyadenylation sites.

In order to attach DNA sequences with regulatory functions, such as promoters, to a CG53135-12 family member gene sequence or to insert a CG53135-12 family member gene sequence into the cloning site of a vector, linkers or adapters providing the appropriate compatible restriction sites may be ligated to the ends of the cDNAs by techniques well known in the art (see e.g., Wu et al., 1987, Methods in Enzymol, 152:343-349). Cleavage with a restriction enzyme can be followed by modification to create blunt ends by digesting back or filling in single-stranded DNA termini before ligation. Alternatively, a desired restriction enzyme site can be introduced into a fragment of DNA by amplification of the DNA using PCR with primers containing the desired restriction enzyme site.

An expression construct comprising a CG53135-12 family member sequence operably associated with regulatory regions can be directly introduced into appropriate host cells for expression and production of a CG53135-12 family protein without further cloning. See, e.g., U.S. Pat. No. 5,580,859. The expression constructs can also contain DNA sequences that facilitate integration of a CG53135-12 family member sequence into the genome of the host cell, e.g., via homologous recombination. In this instance, it is not necessary to employ an expression vector comprising a replication origin suitable for appropriate host cells in order to propagate and express a CG53135-12 family member in the host cells.

A variety of expression vectors may be used, including but are not limited to, plasmids, cosmids, phage, phagemids or modified viruses. Such host-expression systems represent vehicles by which the coding sequences of a CG53135-12 family member gene may be produced and subsequently purified, but also represent cells which may, when transformed or transfected with the appropriate nucleotide coding sequences, express a CG53135-12 family member in situ. These include, but are not limited to, microorganisms such as bacteria (e.g., E. coli and B. subtilis) transformed with recombinant bacteriophage DNA, plasmid DNA or cosmid DNA expression vectors containing CG53135-12 family member coding sequences; yeast (e.g., Saccharomyces, Pichia) transformed with recombinant yeast expression vectors containing CG53135 coding sequences; insect cell systems infected with recombinant virus expression vectors (e.g., baculovirus) containing CG53135-12 family member coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing CG53135-12 family member coding sequences; or mammalian cell systems (e.g., COS, CHO, BHK, 293, NSO, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli and eukaryotic cells are used for the expression of a recombinant CG53135-12 family molecule. For example, mammalian cells such as Chinese hamster ovary cells (CHO) can be used with a vector bearing promoter element from major intermediate early gene of cytomegalocirus for effective expression of a CG53135-12 family member sequence (Foecking et al., 1986, Gene 45:101; and Cockett et al., 1990, Bio/Technology 8:2).

In bacterial systems, a number of expression vectors may be advantageously selected depending upon the use intended for a CG53135-12 family member being expressed. For example, when a large quantity of a CG53135-12 family member is to be produced, for the generation of pharmaceutical compositions of a CG53135-12 family member, vectors that direct the expression of high levels of readily purified fusion protein products may be desirable. Such vectors include, but are not limited to, the E. coli expression vector pCR2.1 TOPO (Invitrogen); pIN vectors (Inouye & Inouye, 1985, Nucleic Acids Res. 13:3101-3109; Van Heeke & Schuster, 1989, J. Biol. Chem. 24:5503-5509) and the like. Series of vectors like pFLAG (Sigma), pMAL (NEB), and pET (Novagen) may also be used to express the foreign proteins as fusion proteins with FLAG peptide, malE-, or CBD- protein. These recombinant proteins may be directed into periplasmic space for correct folding and maturation. The fused part can be used for affinity purification of the expressed protein. Presence of cleavage sites for specific proteases like enterokinase allows a CG53135-12 family member protein to be cleaved from the fusion protein. The pGEX vectors may also be used to express foreign proteins as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, many vectors to express foreign genes can be used, e.g., Autographa californica nuclear polyhedrosis virus (AcNPV) can be used as a vector to express foreign genes. The virus grows in cells like Spodoptera frugiperda cells. A CG53135 coding sequence may be cloned individually into non-essential regions (e.g., the polyhedrin gene) of the virus and placed under control of an AcNPV promoter (e.g., the polyhedrin promoter).

In mammalian host cells, a number of viral-based expression systems may be utilized. In cases where an adenovirus is used as an expression vector, a CG53135-12 family member coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing CG53135 in infected hosts (see, e.g., Logan & Shenk, 1984, Proc. Natl. Acad. Sci. USA 8 1:355-359). Specific initiation signals may also be required for efficient translation of inserted a CG53135-12 family member coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript and post-translational modification of the gene product, e.g., glycosylation and phosphorylation of the gene product, may be used. Such mammalian host cells include, but are not limited to, PC12, CHO, VERY, BHK, Hela, COS, MDCK, 293, 3T3, W138, BT483, Hs578T, HTB2, BT2O, and T47D, NS0 (a murine myeloma cell line that does not endogenously produce any immunoglobulin chains), CRL7030 and HsS78Bst cells. Expression in a bacterial or yeast system can be used if post-translational modifications turn to be non-essential for a desired activity of CG53135-12 family member. In a preferred embodiment, E. coli is used to express a CG53135-12 family member sequence.

For long-term, high-yield production of properly processed CG53135-12 family member, stable expression in cells is preferred. Cell lines that stably express a CG53135-12 family member may be engineered by using a vector that contains a selectable marker. By way of example but not limitation, following the introduction of the expression constructs, engineered cells may be allowed to grow for 1-2 days in an enriched media, and then are switched to a selective media. The selectable marker in the expression construct confers resistance to the selection and optimally allows cells to stably integrate the expression construct into their chromosomes and to grow in culture and to be expanded into cell lines. Such cells can be cultured for a long period of time while a CG53135-12 family member is expressed continuously.

A number of selection systems may be used, including but not limited to, antibiotic resistance (markers like Neo, which confers resistance to geneticine, or G418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62: 191-217; May, 1993, TIB TECH 11(5):155-2 15); Zeo, for resistance to Zeocin; Bsd, for resistance to blasticidin, etc.); antimetabolite resistance (markers like Dhfr, which confers resistance to methotrexate, Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); and hygro, which confers resistance to hygromycin (Santerre et al., 1984, Gene 30:147). In addition, mutant cell lines including, but not limited to, tk-, hgprt- or aprt- cells, can be used in combination with vectors bearing the corresponding genes for thymidine kinase, hypoxanthine, guanine- or adenine phosphoribosyltransferase. Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150:1.

The recombinant cells may be cultured under standard conditions of temperature, incubation time, optical density and media composition. However, conditions for growth of recombinant cells may be different from those for expression of a CG53135-12 family member. Modified culture conditions and media may also be used to enhance production of a CG53135-12 family member. Any techniques known in the art may be applied to establish the optimal conditions for producing a CG53135-12 family member.

An alternative to producing a CG53135-12 family member or a fragment thereof by recombinant techniques is peptide synthesis. For example, an entire protein of a CG53135-12 family member, or a protein corresponding to a portion of a CG53135-12 family member, can be synthesized by use of a peptide synthesizer. Conventional peptide synthesis or other synthetic protocols well known in the art may be used.

Proteins having the amino acid sequence of a CG53135-12 family member or a portion thereof may be synthesized by solid-phase peptide synthesis using procedures similar to those described by Merrifield, 1963, J. Am. Chem Soc., 85:2149. During synthesis, N-protected amino acids having protected side chains are added stepwise to a growing protein chain linked by its C-terminal and to an insoluble polymeric support, i.e., polystyrene beads. The proteins are synthesized by linking an amino group of an N-α-deprotected amino acid to an α-carboxyl group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc, which is acid-labile, and Fmoc, which is base-labile. Details of appropriate chemistries, resins, protecting groups, protected amino acids and reagents are well known in the art and so are not discussed in detail herein (See, Atherton et al., 1989, Solid Phase Peptide Synthesis: A Practical Approach, IRL Press, and Bodanszky, 1993, Peptide Chemistry, A Practical Textbook, 2nd Ed., Springer-Verlag).

Purification of the resulting CG53135-12 family member is accomplished using conventional procedures, such as preparative HPLC using gel permeation, partition and/or ion exchange chromatography. The choice of appropriate matrices and buffers are well known in the art and so are not described in detail herein.

Non-limiting examples of methods for preparing CG53135-12 can be found in Section 6, infra.

Structural Prediction and Functional Analysis of CG53135-12

Any methods known in the art can be used to determine the identity of a purified CG53135-12 family protein of the instant invention. Such methods include, but are not limited to, Western Blot, sequencing (e.g., Edman sequencing), liquid chromatography (e.g., HPLC, RP-HPLC with both UV and electrospray mass spectrometric detection), mass spectrometry, total amino acid analysis, peptide mapping, and SDS-PAGE. The secondary, tertiary and/or quaternary structure of a CG53135-12 family member protein can analyzed by any methods known in the art, e.g., far UV circular dichroism spectrum can be used to analyze the secondary structure, near UV circular dichroism spectroscopy and second derivative UV absorbance spectroscopy can be used to analyze the tertiary structure, and light scattering SEC-HPLC can be used to analyze quaternary structure.

The purity of a CG53135-12 family protein of the instant invention can be analyzed by any methods known in the art, such as but not limited to, sodium dodecyl sulphate polyacrylamide gel electrophoresis (“SDS-PAGE”), reversed phase high-performance liquid chromatography (“RP-HPLC”), size exclusion high-performance liquid chromatography (“SEC-HPLC”), and Western Blot (e.g., host cell protein Western Blot). In a preferred embodiment, a CG53135-12 family protein in a composition used in accordance to the instant invention is 80%-100% pure by densitometry, or at least 97%, at least 98%, or at least 99% pure by densitometry. In another preferred embodiment, a CG53135-12 family protein in a composition used in accordance to the instant invention is more than 97%, more than 98%, or more than 99% pure by densitometry.

The biological activities and/or potency of a CG53135-12 family member of the present invention can be determined by any methods known in the art. For example, compositions for use in therapy in accordance to the methods of the present invention can be tested in suitable cell lines for one or more activities that CG53135-12 possesses (e.g., cellular proliferation stimulatory activity). Non-limiting examples of such assays are described in Section 6, infra.

Structure prediction, analysis of crystallographic data, sequence alignment, as well as homology modeling, can also be accomplished using computer software programs available in the art, such as BLAST, CHARMm release 21.2 for the Convex, and QUANTA v.3.3, (Molecular Simulations, Inc., York, United Kingdom). Other methods of structural analysis can also be employed. These include, but are not limited to, X-ray crystallography (Engstom, A., 1974, Biochem. Exp. Biol. 11:7-13) and computer modeling (Fletterick, R. and Zoller, M. (eds.), 1986, Computer Graphics and Molecular Modeling, in Current Communications in Molecular Biology, Cold Spring Harbor Laboratory, Cold Spring Harbor, N.Y.).

The half life of a protein is a measurement of protein stability and indicates the time necessary for a one half reduction in activity of the protein. The half-life of a CG53135-12 family member protein can be determined by any method measuring activity of a CG53135-12 family member in samples from a subject over a period of time. The normalization to concentration of CG53135-12 family member in the sample can be done by, e.g., immunoassays using anti-CG53135 antibodies to measure the levels of the a CG53135-12 family member molecules in samples taken over a period of time after administration of the CG53135-12 family member, or detection of radiolabelled CG53135-12 family member in samples taken from a subject after administration of the radiolabeled CG53135-12 family member. In specific embodiments, techniques known in the art can be used to prolong the half life of a CG53135-12 family member in vivo. For example, albumin or inert polymer molecules such as high molecular weight polyethyleneglycol (PEG) can be used. See, e.g., International Publication Nos. WO 93/15199, WO 93/15200, and WO 01/77137; and U.S. Pat. No. 6,528,485.

Compositions comprising one more CG53135-12 family members for use in a therapy can also be tested in suitable animal model systems prior to testing in humans. To establish an estimate of drug activity in relevant model experiments, an index can be developed that combines observational examination of the animals as well as their survival status. The effectiveness of a CG53135-12 family member on preventing and/or treating a disease can be monitored by any methods known to one skilled in the art, including but not limited to, clinical evaluation, and measuring the level of CG53135 biomarkers in a biosample.

Any adverse effects during the use of a CG53135-12 family member alone or in combination with another therapy (e.g., another therapeutic or prophylactic agent) are preferably also monitored. Examples of adverse effects of administering a CG53135 protein include, but are not limited to, nausea; chills; fever, vomiting; dizziness; photopsia (vision-“lights flashing”) and astigmatism (mild astigmatism); neuropathy (on soles of the feet); tachycardia; headache; and asymptomatic, and single premature atrial complex noted on ECG. Undesired effects typically experienced by patients taking one or more agents other than CG53135 are numerous and known in the art. Many are described in the Physicians' Desk Reference (58th ed., 2004).

Therapeutic and Prophylactic Uses of CG53135-12

The present invention provides nucleic acids and proteins of CG53135-12 and its fragments, derivatives, variants, homologs, analogs, and their uses in stimulating proliferation of epithelial cells and/or mesenchymal cells. Accordingly, a CG53135-12 family member may also be used in wound and/or burn repairing and healing, ligament repairing, cartilage growth and/or repairing, promoting skin graft growth, increasing bone density, stimulating stem cell growth and/or differentiation, preventing and/or treating stroke, Alzheimer's disease, ischemic heart disease and/or aneurysms, or ulcers. CG53135-12 family members can be used in indications where FGF-20 has been show to be effective, such as those described in, e.g., U.S. patent application Ser. Nos. 10/435,087, filed May 9, 2003 (preventing and/or treating oral mucositis), 09/992,840, filed Nov. 6, 2001, 10/011,364, filed Nov. 16, 2001, and 10/321,962, filed Dec. 16, 2002 (preventing and/or treating inflammatory bowel disease (“IBD”)), 10/842,206, filed May 10, 2004 (preventing and/or treating arthritis and/or certain diseases related to central nerve system, such as Parkinson's Disease, and certain diseases related to cardiovascular system, such as stroke); and 10/842,179, filed May 10, 2004 (preventing and/or treating a disorder or symptom associated with radiation exposure). The content of each reference is incorporated herein by reference in its entirety.

Toxicity and therapeutic efficacy of a composition of the invention (e.g., a composition comprising one or more CG53135-12 family proteins) can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio of LD50/ED50. Compositions that exhibit large therapeutic indices are preferred. While compositions that exhibit toxic side effects may be used, care should be taken to design a delivery system that targets such composition to the site of affected tissue in order to minimize potential damage to uninfected cells and, thereby, reduce side effects.

In one embodiment, the data obtained from the cell culture assays and animal studies can be used in formulating a range of dosage for use in humans. The dosage of complexes lies preferably within a range of circulating concentrations that include the ED50 with little or no toxicity. The dosage may vary within this range depending upon the dosage form employed, the route of administration utilized, the severity of the disease, age and weight of the subject, and other factors normally considered by a medical professional (e.g., a physician). For any composition used in the method of the invention, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell cultures. Such information can be used to more accurately determine useful doses in humans. Levels in plasma may be measured, for example, by high performance liquid chromatography.

The amount of the composition of the invention which will be effective in the treatment of a particular disorder or condition will depend on the nature of the disorder or condition, and can be determined by standard clinical techniques. The precise dose to be employed in the formulation will also depend on the route of administration, and the seriousness of the disease or disorder, and should be decided according to the judgment of the practitioner and each patient's circumstances.

In one embodiment, the dosage of a composition comprising one or more CG53135-12 family proteins for administration in a human patient provided by the present invention is at least 0.001 mg/kg, at least 0.005 mg/kg, at least 0.01 mg/kg, at least 0.03 mg/kg, at least 0.05 mg/kg, at least 0.1 mg/kg, at least 0.2 mg/kg, at least 0.3 mg/kg, at least 0.4 mg/kg, at least 0.5 mg/kg, at least 0.6 mg/kg, at least 0.7 mg/kg, at least 0.8 mg/kg, at least 0.9 mg/kg, at least 1 mg/kg, at least 2 mg/kg, at least 3 mg/kg, at least 4 mg/kg, at least 5 mg/kg, at least 6 mg/kg, at least 7 mg/kg, at least 8 mg/kg, at least 9 mg/kg, at least 10 mg/kg, at least 25 mg/kg, at least 50 mg/kg, at least 75 mg/kg, or at least 100 mg/kg (as measured by UV assay). In another embodiment, the dosage of a composition comprising one or more CG53135 proteins for administration in a human patient provided by the present invention is between 0.001-100 mg/kg, between 0.001-50 mg/kg, between 0.001-25 mg/kg, between 0.001-10 mg/kg, between 0.005-5 mg/kg, between 0.01-1 mg/kg, between 0.01-0.9 mg/kg, between 0.01-0.8 mg/kg, between 0.01-0.7 mg/kg, between 0.01-0.6 mg/kg, between 0.01-0.5 mg/kg, or between 0.01-0.3 mg/kg (as measured by UV assay).

Protein concentration can be measured by methods known in the art, such as Bradford assay or UV assay, and the concentration may vary depending on what assay is being used. In a non-limiting example, the protein concentration in a pharmaceutical composition of the instant invention is measured by a UV assay that uses a direct measurement of the UV absorption at a wavelength of 280 nm, and calibration with a well characterized reference standard of CG53135 protein (instead of IgG). Test results obtained with this UV method (using CG53135 reference standard) are three times lower than test results for the same sample(s) tested with the Bradford method (using IgG as calibrator). For example, if a dosage of a composition comprising one or more CG53135 proteins for administration in a human patient provided by the present invention is between 0.001-10 mg/kg measured by UV assay, then the dosage is 0.003-30 mg/kg as measured by Bradford assay.

The appropriate and recommended dosages, formulation and routes of administration for treatment modalities such as chemotherapeutic agents, radiation therapy and biological/immunotherapeutic agents such as cytokines, which can be used in combination with a composition comprising one or more CG53135-12 family member, are known in the art and described in such literature as the Physician's Desk Reference (58th ed., 2004).

The present invention also provides methods of identifying individuals who are carriers of the genetic risk-altering factor for a decreased risk of osteoarthritis, and therefore have a decreased risk for osteoarthritis and resultant musculoskeletal complications.

The present invention further provides the use of CG53135-12 or a haplotype containing CG53135-12 to designate the FGF-20 protein as a target for pharmaceutical intervention in elevated risk of osteoarthritis, and as an aid in the design, testing, or evaluation of such pharmaceutical compounds.

Pharmaceutical Compositions

Various delivery systems are known and can be used to administer a composition used in accordance to the methods of the invention. Such delivery systems include, but are not limited to, encapsulation in liposomes, microparticles, microcapsules, expression by recombinant cells, receptor-mediated endocytosis, construction of the nucleic acids of the invention as part of a retroviral or other vectors, etc. Methods of introduction include, but are not limited to, intradermal, intramuscular, intraperitoneal, intrathecal, intracerebroventricular, epidural, intravenous, subcutaneous, intranasal, intratumoral, transdermal, transmucosal, rectal, and oral routes. The compositions used in accordance to the methods of the invention may be administered by any convenient route, for example, by infusion or bolus injection, by absorption through epithelial or mucocutaneous linings (e.g., eye mucosa, oral mucosa, vaginal mucosa, rectal and intestinal mucosa, etc.), and may be administered together with other biologically active agents. Administration can be systemic or local. In a specific embodiment, the present invention comprises using single or double chambered syringes, preferably equipped with a needle-safety device and a sharper needle, that are pre-filled with a composition comprising one or more CG53135 proteins. In one embodiment, dual chambered syringes (e.g., Vetter Lyo-Ject dual-chambered syringe by Vetter Pharmar-Fertigung) are used. Such systems are desirable for lyophilized formulations, and are especially useful in an emergency setting.

In some embodiments, it may be desirable to administer the pharmaceutical compositions of the invention locally to the area in need of treatment. This may be achieved by, for example, local infusion during surgery, or topical application, e.g., in conjunction with a wound dressing after surgery, by injection, by means of a catheter, by means of a suppository, or by means of an implant (said implant being of a porous, non-porous, or gelatinous material, including membranes, such as sialastic membranes, or fibers). In one embodiment, administration can be by direct injection at the site (or former site) of rapidly proliferating tissues that are most sensitive to an insult, such as radiation, chemotherapy, or chemical/biological warfare agent.

In some embodiments, where the composition of the invention is a nucleic acid encoding a prophylactic or therapeutic agent, the nucleic acid can be administered in vivo to promote expression of their encoded proteins (e.g., CG53135-12 family proteins), by constructing the nucleic acid as part of an appropriate nucleic acid expression vector and administering it so that it becomes intracellular, e.g., by use of a retroviral vector, or by direct injection, or by use of microparticle bombardment (e.g., a gene gun), or coating with lipids or cell-surface receptors or transfecting agents, or by administering it in linkage to a homeobox-like peptide which is known to enter the nucleus, etc. Alternatively, a nucleic acid of the invention can be introduced intracellularly and incorporated within host cell DNA for expression, by homologous recombination.

The instant invention encompasses bulk drug compositions useful in the manufacture of pharmaceutical compositions that can be used in the preparation of unit dosage forms. In a preferred embodiment, a composition of the invention is a pharmaceutical composition. Such compositions comprise a prophylactically or therapeutically effective amount of CG53135, and a pharmaceutically acceptable carrier. Preferably, the pharmaceutical compositions are formulated to be suitable for the route of administration to a subject.

In one embodiment, the term “pharmaceutically acceptable” means approved by a regulatory agency of the Federal or a state government or listed in the U.S. Pharmacopeia or other generally regarded as safe for use in humans (GRAS). The term “carrier” refers to a diluent, adjuvant, bulking agent (e.g., arginine in various salt forms, sulfobutyl ether Beta-cyclodextrin sodium, or sucrose), excipient, or vehicle with which CG53135 is administered. Such pharmaceutical carriers can be sterile liquids, such as water and oils (e.g., oils of petroleum, animal, vegetable or synthetic origins, such as peanut oil, soybean oil, mineral oil, sesame oil and the like), or solid carriers, such as one or more substances which may also act as diluents, flavoring agents, solubilizers, lubricants, suspending agents, or encapsulating material. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include, but are not limited to, starch or its synthetically modified derivatives such as hydroxyethyl starch, stearate salts, glycerol, glucose, lactose, sucrose, trehalose, gelatin, sulfobutyl ether Beta-cyclodextrin sodium, sodium chloride, glycerol, propylene, glycol, water, ethanol, or a combination thereof. The composition, if desired, can also contain minor amounts of wetting or emulsifying agents, or pH buffering agents.

The compositions comprising one or more CG53135-12 family members may be formulated into any of many possible dosage forms such as, but not limited to, liquid, suspension, microemulsion, microcapsules, tablets, capsules, gel capsules, soft gels, pills, powders, enemas, sustained-release formulations and the like. The compositions comprising a CG53135-12 family member may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. The composition can also be formulated as a suppository, with traditional binders and carriers such as triglycerides. Oral formulation can include standard carriers, such as pharmaceutical grades of mannitol, lactose, starch or its synthetically modified derivatives such as hydroxyethyl starch, stearate salts, sodium saccharine, cellulose, magnesium carbonate, etc.

A pharmaceutical composition comprising one or more CG53135-12 family members is formulated to be compatible with its intended route of administration. In a specific embodiment, the composition is formulated in accordance with routine procedures as a pharmaceutical composition adapted for intravenous, subcutaneous, intramuscular, oral, intranasal, intratumoral or topical administration to human beings. Typically, compositions for intravenous administration are solutions in sterile isotonic or hypertonic aqueous buffer. Where necessary, the composition may also include a solubilizing agent and a local anesthetic such as benzyl alcohol or lidocaine to ease pain at the site of the injection.

If a composition comprising one or more CG53135-12 family members is to be administered topically, the composition can be formulated in the form of transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Preferred topical formulations include those in which the compositions of the invention are in admixture with a topical delivery agent, such as but not limited to, lipids, liposomes, fatty acids, fatty acid esters, steroids, chelating agents and surfactants. The compositions comprising one or more CG53135-12 family members may be encapsulated within liposomes or may form complexes thereto, in particular to cationic liposomes. Alternatively, the compositions comprising CG53135 may be complexed to lipids, in particular to cationic lipids. For non-sprayable topical dosage forms, viscous to semi-solid or solid forms comprising a carrier or one or more excipients compatible with topical application and having a dynamic viscosity preferably greater than water are typically employed. Other suitable topical dosage forms include sprayable aerosol preparations wherein the active ingredient, preferably in combination with a solid or liquid inert carrier, is packaged in a mixture with a pressurized volatile (e.g., a gaseous propellant, such as Freon or hydrofluorocarbons) or in a squeeze bottle. Moisturizers or humectants can also be added to pharmaceutical compositions and dosage forms if desired. Examples of such additional ingredients are well-known in the art.

A composition comprising one or more CG53135-12 family members can be formulated in an aerosol form, spray, mist or in the form of drops or powder if intranasal administration is preferred. In particular, a composition comprising one or more CG53135-12 family members can be conveniently delivered in the form of an aerosol spray presentation from pressurized packs or a nebulizer, with the use of a suitable propellant (e.g., dichlorodifluoromethane, trichlorofluoromethane, dichlorotetrafluoroethane, other hydrofluorocarbons, carbon dioxide or other suitable gas). In the case of a pressurized aerosol the dosage unit may be determined by providing a valve to deliver a metered amount. Microcapsules (composed of, e.g., polymerized surface) for use in an inhaler or insufflator may be formulated containing a powder mix of the compound and a suitable powder base such as dissacharides or starch.

One or more CG53135-12 family proteins may also be formulated into a microcapsule with one or more polymers (e.g., hydroxyethyl starch) form the surface of the microcapsule. Such formulations have benefits such as slow-release.

A composition comprising one or more CG53135-12 family members can be formulated in the form of powders, granules, microparticulates, nanoparticulates, suspensions or solutions in water or non-aqueous media, capsules, gel capsules, sachets, tablets or minitablets if oral administration is preferred. Thickeners, flavoring agents, diluents, emulsifiers, dispersing aids or binders may be desirable. Tablets or capsules can be prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods well-known in the art. Liquid preparations for oral administration may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of a prophylactic or therapeutic agent(s).

In one embodiment, the compositions of the invention are orally administered in conjunction with one or more penetration enhancers, e.g., alcohols, surfactants and chelators. Preferred surfactants include, but are not limited to, fatty acids and esters or salts thereof, bile acids and salts thereof. In some embodiments, combinations of penetration enhancers are used, e.g., alcohols, fatty acids/salts in combination with bile acids/salts. In a specific embodiment, sodium salt of lauric acid, capric acid is used in combination with UDCA. Further penetration enhancers include, but are not limited to, polyoxyethylene-9-lauryl ether, polyoxyethylene-20-cetyl ether. Compositions of the invention may be delivered orally in granular form including, but is not limited to, sprayed dried particles, or complexed to form micro or nanoparticles. Complexing agents that can be used for complexing with the compositions of the invention include, but are not limited to, poly-amino acids, polyimines, polyacrylates, polyalkylacrylates, polyoxethanes, polyalkylcyanoacrylates, cationized gelatins, albumins, acrylates, polyethyleneglycols (PEG), DEAE-derivatized polyimines, pollulans, celluloses, and starches. Particularly preferred complexing agents include, but are not limited to, chitosan, N-trimethylchitosan, poly-L-lysine, polyhistidine, polyornithine, polyspermines, protamine, polyvinylpyridine, polythiodiethylamino-methylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG).

A composition comprising one or more CG53135-12 family members can be delivered to a subject by pulmonary administration, e.g., by use of an inhaler or nebulizer, of a composition formulated with an aerosolizing agent.

In a preferred embodiment, a composition comprising one or more CG53135-12 family members is formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle (e.g., sterile pyrogen-free water) before use.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as benzyl alcohol or lidocaine to ease pain at the site of the injection. Generally, the ingredients are supplied either separately or mixed together in unit dosage form, for example, as a dry lyophilized powder or water free concentrate in a sealed container, such as a vial, ampoule or sachette, indicating the quantity of active agent. Where the composition is to be administered by infusion, it can be dispensed with an infusion container containing sterile pharmaceutical grade water or saline. Where the composition is administered by injection, an ampoule or vial of sterile water for injection or saline can be provided so that the ingredients may be mixed prior to administration.

A composition comprising one or more CG53135-12 family members can be formulated as neutral or salt forms. Pharmaceutically acceptable salts include, but are not limited to, those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

In addition to the formulations described previously, a composition comprising one or more CG53135-12 family members may also be formulated as a depot preparation. Such long acting formulations may be administered by implantation (for example, subcutaneously or intramuscularly) or by intramuscular injection. Thus, for example, the compositions may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

In one embodiment, the ingredients of the compositions used in accordance to the methods of the invention are derived from a subject that is the same species origin or species reactivity as recipient of such compositions.

In some embodiments, a formulation used in accordance to the methods of the invention comprises 0.02 M-0.2 M acetate, 0.5-5% glycerol, 0.2-0.5 M arginine-HCl, and one or more CG53135-12 family proteins, preferably 0.5-5 mg/ml (UV). In one embodiment, a formulation used in accordance to the methods of the invention comprises 0.04M sodium acetate, 3% glycerol (volume/volume), 0.2 M arginine-HCl at pH 5.3, and one or more isolated CG53135 proteins, preferably 0.8 mg/ml (UV). In some embodiments, a formulation used in accordance to the methods of the invention comprises 0.01-1 M of a stabilizer, such as arginine in various salt forms, sulfobutyl ether Beta-cyclodextrin sodium, or sucrose, 0.01-0.1 M sodium phosphate monobasic (NaH2PO4.H2O), 0.01%-0.1% weight/volume (“w/v”) polysorbate 80 or polysorbate 20, and one or more CG53135-12 family proteins, preferably 0.005-50 mg/ml (UV). In one embodiment, a formulation used in accordance to the methods of the invention comprises 30 mM sodium citrate, pH 6.1, 2 mM EDTA, 200 mM sorbitol, 50 mM KCl, 20% glycerol, and one or more isolated CG53135-12 family proteins.

The invention also provides kits for carrying out the therapeutic regimens of the invention. Such kits comprise in one or more containers prophylactically or therapeutically effective amounts of the composition of the invention (e.g., a composition comprising one or more CG53135 proteins) in pharmaceutically acceptable form. The composition in a vial of a kit of the invention may be in the form of a pharmaceutically acceptable solution, e.g., in combination with sterile saline, dextrose solution, or buffered solution, or other pharmaceutically acceptable sterile fluid. Alternatively, the composition may be lyophelized or desiccated; in this instance, the kit optionally further comprises in a container a pharmaceutically acceptable solution (e.g., saline, dextrose solution, etc.), preferably sterile, to reconstitute the composition to form a solution for injection purposes.

In another embodiment, a kit of the invention further comprises a needle or syringe, preferably packaged in sterile form, for injecting the formulation, and/or a packaged alcohol pad. Instructions are optionally included for administration of the formulations of the invention by a clinician or by the patient.

In some embodiments, the present invention provides kits comprising a plurality of containers each comprising a pharmaceutical formulation or composition comprising a dose of the composition of the invention (e.g., a composition comprising one or more CG53135 proteins) sufficient for a single administration.

As with any pharmaceutical product, the packaging material and container are designed to protect the stability of the product during storage and shipment. In one embodiment, compositions of the invention are stored in containers with biocompatible detergents, including but not limited to, lecithin, taurocholic acid, and cholesterol; or with other proteins, including but not limited to, gamma globulins and serum albumins. Further, the products of the invention include instructions for use or other informational material that advise the physician, technician, or patient on how to appropriately prevent or treat the disease or disorder in question.

### EXAMPLES

The present invention is further illustrated by the following non-limiting examples.

### Example 1

### Identification of Novel SNP Variant of FGF-20

The invention relates to the identification of a polymorphism in the gene encoding FGF-20 (SEQ ID NO:4) in humans. The SNP variant is designated CG53135-12.

### Method of Novel SNP Identification and Confirmation

CG53135-12 was identified as a novel Single nucleotide polymorphism (SNP) of FGF-20 (SEQ ID NOs:3 and 4)

SeqCalling™ Technology: cDNA was derived from various human samples representing multiple tissue types, normal and diseased states, physiological states, and developmental states from different donors. Samples were obtained as whole tissue, cell lines, primary cells or tissue cultured primary cells and cell lines. Cells and cell lines may have been treated with biological or chemical agents that regulate gene expression for example, growth factors, chemokines, and/or steroids. The cDNA thus derived was then sequenced using CuraGen Corporation's proprietary SeqCalling™ technology. Sequence traces were evaluated manually and edited for corrections if appropriate. cDNA sequences from all samples were assembled with themselves and with public ESTs using bioinformatics programs to generate CuraGen Corporation's human SeqCalling database of SeqCalling assemblies. Each assembly contains one or more overlapping cDNA sequences derived from one or more human samples. Fragments and ESTs were included as components for an assembly when the extent of identity with another component of the assembly was at least 95% over 50 bp. Each assembly can represent a gene and/or its variants such as splice forms and/or single nucleotide polymorphisms (SNPs) and their combinations.

Method of novel SNP Identification: SNPs are identified by analyzing SeqCalling sequence assemblies using CuraGen Corporation's proprietary SNPTool™ algorithm SNPTool™ identifies variation in assemblies with the following criteria: SNPs are not analyzed within 10 base pairs on both ends of an alignment; Window size (number of bases in a view) is 10; The allowed number of mismatches in a window is 2; Minimum SNP base quality (PHRED score) is 23; Minimum number of changes to score an SNP is 2/assembly position. SNPTool™ analyzes the assembly and displays SNP positions, associated individual variant sequences in the assembly, the depth of the assembly at that given position, the putative assembly allele frequency, and the SNP sequence variation. Sequence traces are then selected and brought into view for manual validation. The consensus assembly sequence is imported into CuraTools™ along with variant sequence changes to identify potential amino acid changes resulting from the SNP sequence variation. Comprehensive SNP data analysis is then exported into the SNPCalling database.

Method of novel SNP Confirmation: SNPs are confirmed employing one of several validated methods, for instance Pyrosequencing (Pyrosequencing, Sweden), MassArray (Sequenom, San Diego, USA), or BeadArray (Illumina, San Diego, USA).

TABLE 2A
Summary table of SNP CG53135-12 (nucleotide)

| | Nucleotides | | |
|---|---|---|---|
| Variant | Position | Initial | Modified |
| CG53135-12 | 616 | G | A |

TABLE 2B
Summary table of SNP CG53135-12 (amino acids)

| | Amino Acids | | | |
|---|---|---|---|---|
| Variant | Position | Initial | Modified | Change type |
| CG53135-12 | 206 | Asp | Asn | Acidic to Uncharged |
| | | | | Polar |

### Statistical Analysis for Each Marker/Trait Combination

An individual was defined as informative if both the trait value and genotype were available. The markers we tested were all bi-allelic.

Hardy-Weinberg tests: Hardy-Weinberg equilibrium (HWE) relates genotype frequencies to allele frequencies under general assumptions of an equilibrium population. Violations of HWE may indicate selection against the minor allele and population stratification.

Selection against the minor allele occurs when the minor allele detracts from evolutionary fitness and may result in having fewer homozygotes than would be expected by chance.

To perform Hardy-Weinberg tests, the counts of individuals with AA, AB, and BB genotypes in this population were termed N(AA), N(AB), and N(BB), respectively, and the allele frequency p was calculated as

p=[N(AA)+0.5N(BB)]/N.

Next, the counts of individuals expected for each genotype under the null hypothesis of HWE were calculated as

n(AA)=p²N

n(AB)=2pqN

n(BB)=q²N

Finally, two test statistics were calculated:

HW1=[N(AA)−n(AA)]²/n(AA)+[N(AB)−n(AB))]²/n(AB)+[N(BB)−n(BB)]²/n(BB)

HW2={[N(AA)+N(BB)]−[n(AA)+n(BB)]}²/{n(AA)+n(BB)}+[N(AB)−N(AB)]²/n(AB)

Under the null hypothesis, both HW1 and HW2 follow χ distributions with 1 degree of freedom. The critical values of χ² for p-values of 0.05 and 0.01 are 3.84 and 6.63 respectively. Values of χ² larger than these indicate a 5% chance or a 1% chance of the HW assumptions being satisfied.

The HW1 test is the standard test, but it is not accurate when the smallest category, typically N(AA), has fewer than 5 individuals. The HW2 test is more robust but can be less sensitive for rare alleles.

If there is significant deviation from HWE, the sign of [N(AA)+N(BB)]−[n(AA)+n(BB)] indicates the reason: positive values indicate stratification and negative values indicate selection against the minor allele.

Association tests were based on a case-control model. Standard statistical tests have been incorporated into GeneScape and validated. Results presented are by Fisher's exact two-tailed test, with cell counts collapsed for a dominant effect of the minor allele (i.e. minor allele homozygotes and heterozygotes were combined for the Osteoarthritis and Unaffected categories).

### Example 2

### Genetic Population Tested

Suitable cases were recruited into the study by sampling 2000 index cases from a knee replacement register. All subjects gave informed consent.

Knee OA case inclusion criteria:

1) Caucasian, Male or female, aged 43-92 yrs.

2) Definite radiographic OA of at least one compartment (patello-femoral and or tibiofemoral) seen on a standardised anterior-posterior (AP) standing (fully extended) and skyline or lateral (30 degree flexion) radiographs. Definite OA required joint space narrowing (grade 2 or more) plus osteophytes (grade 1 or more) assessed using a standard atlas. In addition, all radiographic features of OA were scored separately for each of the three compartments. Subjects who had undergone surgical replacement for OA were included into the study provided their pre-operative radiographs confirmed definite OA status.

3) However, the following subjects were excluded from the study:

a. Subjects with previous meniscectomy or fracture at the knee that may have resulted in ‘secondary’ knee OA.

b. Subjects with preceding or concurrent history of joint disease (e.g., juvenile chronic arthritis, TB, sepsis, haemophilia, rheumatoid, psoriatic or other seronegative spondarthropathy) that may have resulted in knee damage.

4) Control subjects are sampled from the same base population. Both the case and control subjects are matched for genetic background (Caucasians), age range (43-92 yrs), sex and other demographic factors. Demographic characteristics of the case and control subgroups are therefore broadly similar. Also, sex specific characteristics are similar in the case and control groups.

### Results

Genotyping results showing A and G ALLELES: homozygous major allele, heterozygous, and homozygous minor allele at SNP13375517 (CG53135-12).
Homozygous G/G: 769
Heterozygous A/G: 26
Homozygous A/A: 1

Association results showing distribution of A allele in Osteoarthritis and Normal subjects. P-value is by Fisher's exact case-control test, two-sided.

TABLE 3

| | A allele not | | |
|---|---|---|---|
| | present | A allele present | p value |
| Osteoarthritis | 388 | 5 | .0002 |
| Normal | 381 | 22 | |

The single-nucleotide polymorphism variant of FGF-20 described here (SNP13375517 allele A, i.e., CG53135-12) is significantly associated with decreased risk for osteoarthritis. Carriers of the minor allele are significantly more likely to be protected from Osteoarthritis than are individuals who are homozygous for the major G allele. The resulting variant amino acid sequence is likely to alter the structure of FGF-20 in a manner that reduces its intermolecular dimerization. Hence the A allele of SNP13375517 in FGF-20 may be of use as a target for diagnostic tests or pharmaceutical intervention in osteoarthritis, and as an aid in the design, testing, or evaluation of such pharmaceutical compounds, and in the design.

### Example 3

### Molecular Modeling

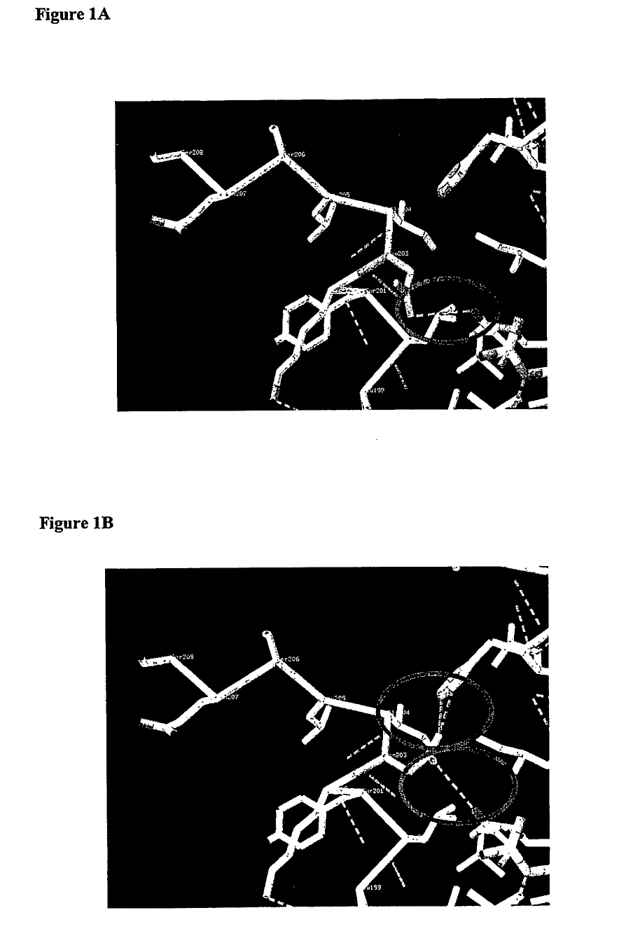

CCG53135-12 is a novel variant of FGF-20 (SEQ ID NOs: 3 and 4) that contains a novel G to A mutation at nucleotide position 616 (Table 2A). CG53135-12 results in an aspartic acid (acidic) to an asparagine (neutral) substitution at amino acid 206 (Table 2B). This SNP is located at the extreme C-terminal end of the molecule. FGF-20 is 68% homologous to FGF-9; however, almost all of the amino acids involved in the dimerization of the two molecules are identical. The human FGF-9 has been crystallized, so biochemical characteristics of FGF-20 can be extrapolated from modeling of FGF-9. As depicted in FIG. 1A, the X-ray crystallographic structure of FGF-9 (see J. Biol. Chem. 2001, 276: 4322-4329) indicates that the substitution of Arginine for Aspartic acid at this position creates additional intramolecular H-bonds with Histidine 56, thereby greatly increasing the structural stability of the C-terminal end of CG53135. The amino acid change in CG53135-12 is also located one position away from leucine 207, which is predicted to be involved in forming homodimers. According to the FGF-9 model, upon dimerization, a significant receptor binding site is occluded. Thus, dimerization of FGF-20 will also likely act in an autinhibitory fashion to decrease interactions with the FGF receptors. Modeling shows that the amino acid substitution in CG53135-12 results in potential structural alterations that may decrease dimerization, thus increasing its interaction with FGF receptors and its subsequent potency as a growth factor (FIG. 1B). Therefore, this SNP is critical to the protein's form or function, as predicted from the crystal structure of FGF-9.

### Example 4

### Receptor Binding Specificity of CG53135 (Study L-116.01)

FGF family members transduce signals intracellularly via high affinity interactions with cell surface immunoglobulin (Ig) domain-containing tyrosine kinase FGF receptors (FGFRs). Four distinct human genes encode FGFRs (See Endocr Relat Cancer 2000, 7:165-97; Front Biosci 1999, 4:D165-77; Cell Tissue Res 1999, 296:3343). A related fifth human sequence lacking a kinase domain has recently been identified and named FGFR-5 (See Biochim Biophys Acta 2001, 1518:152-6). These receptors can each bind several different members of this family (See Omitz et al., J Biol Chem 1996, 271:15292-7). FGFs also bind, albeit with low affinity, to heparin sulfate proteoglycans (HSPGs) present on most cell surfaces and extracellular matrices (ECM). Interactions between FGFs and HSPGs serve to stabilize FGF/FGFR interactions and to sequester FGF and protect it from degradation (See Endocr Relat Cancer 2000, 7:165-97; Int Rev Cytol 1999,185:45-106). Dimerization of FGF receptor monomers upon ligand binding is reported to be a requisite for activation of the kinase domains, leading to receptor trans-phosphorylation. FGF receptor-1 (FGFR-1), which shows the broadest expression pattern of the four FGF receptors, contains at least seven tyrosine phosphorylation sites. A number of signal transduction molecules are affected by binding with different affinities to these phosphorylation sites.

FGFR-1, FGFR-2 and FGFR-3 each recognize FGF-1, FGF-2, FGF-4 and FGF-8. In addition, FGFR-1 & FGFR-2 bind FGF-3, FGF-5, FGF-6, FGF-10 and FGF-17 (See Endocr Relat Cancer 2000, 7:165-97). Binding of various FGF ligands varies with each receptor splice form, thus allowing a wide repertoire of FGF-mediated signaling events through a limited number of receptor coding genes. Tissue-specific alternate splicing permits cells expressing a single FGFR gene to significantly diversify their biological response by generating distinct receptor isoforms that may exhibit different ligand specificity and function. FGFR-4, binds FGF-1, FGF-2, FGF-4, FGF-6, FGF-8 and FGF-9 but not FGF-3, FGF-5 or FGF-7. FGF-7, or keratinocyte growth factor-1 (KGF-1) is only recognized by FGFR-2, whereas FGF-9 binds to FGFR-2, FGFR-3 and FGFR-4. Receptor specificity of FGFs-11 to -19 is not well understood (See Endocr Relat Cancer 2000, 7:165-97; J Biol Chem 1996, 271:15292-7).

Immunohistochemistry studies (Hughes, J Histochem Cytochem 1997, 45:1005-19) in normal human adult tissues from the major organ systems indicated that FGFR-1, FGFR-2 and FGFR-3 are widely expressed, suggesting an important functional role in tissue homeostasis. Protein expression patterns for tissue-specific isoforms have not yet been determined. FGFR-4 has a more limited expression pattern being notably absent from lung, oviduct, placenta, testis, prostate, thyroid, parathyroid, and sympathetic ganglia, tissues where all three other receptors are predominantly expressed (See J Histochem Cytochem 1997, 45:1005-19).

To determine the receptor binding specificity of CG53135-12 and FGF-20 (CG53135-01 or CG53135-05, both encode the protein having the amino acid sequence of SEQ ID NO:4), we examined the effect of soluble FGFRs on the induction of DNA synthesis in NIH 3T3 cells by recombinant FGF-20 (SEQ ID NO: 4) produced in E. coli.

### Materials and Methods

Protein (CG53135-01) Purification from Escherichia coli: For production in E. coli, plasmid pETMY-hFGF20X was transformed into the E. coli expression host BL21 (Novagen, Madison, Wis.) and the induction of protein CG53135 expression was carried out according to the manufacturer's instructions. pETMYhFGF20X/BL21 E. coli bacteria were grown in LB medium at 37° C. At an OD of 0.6, bacteriophage lambda (CE6) was added to a final multiplicity of infection of 5. The infected culture was further incubated at 27° C. for 3 hours. After induction, total cells were harvested, and proteins were analyzed by Western blotting using anti-HisGly antibody (Invitrogen). Cells were harvested by low-speed centrifugation (5000 rpm in a GS-3 rotor for 15 minutes at 4° C.), suspended in phosphate-buffered saline (PBS) containing 0.5M NaCl and 1M arginine, and disrupted with two passes through a microfluidizer. Cell debris was removed by low-speed centrifugation and the soluble protein fraction (supernatant) was clarified by filtration through a 0.2 micron low-protein binding membrane. The protein sample was then loaded onto a metal chelation column (pre-charged with nickel sulfate). The nickel column was washed with PBS/0.5M NaCl+1M L-arginine and bound proteins were eluted with a linear gradient of imidazole (0-0.5 M). Fractions containing CG53135 (100-150 mM imidazole) were pooled and dialyzed against 1×10⁶ volumes of PBS pH 8.0 containing 1M L-arginine. The protein sample was stored at −80° C.

Receptor Specificity: NIH 3T3 cells were cultured in 96-well plates to approximately 100% confluence, washed and fed with DMEM without supplements (Life Technologies), and incubated for 24 h. Recombinant CG53135-01 or control protein was then added to the cells for 18 h. Control proteins used were aFGF (positive control) and platelet derived growth factor-BB (PDGFBB) (negative control). To analyze the effect of soluble FGFRs on CG53135 activity, recombinant CG53135-01, aFGF, or PDGF-BB (final concentrations of 10, 5 and 3 ng/mL, respectively), were mixed with soluble receptors (final concentrations of 0.2, 1 and 5 ug/mL), and incubated for 30 min at 37° C. prior to addition to serum-starved NIH 3T3 cells. Factor concentrations represent the amount of ligand needed to generate a half maximal BrdU response in NIH 3T3 cells. Soluble FGFRs were Fc chimeras of the following receptor forms (FGFR1β (IIIc); FGFR2β (IIIb); FGFR2α (IIIb); FGFR2α (III); FGFR3α (IIIc); FGFR4) and were obtained from R&D Systems (Minneapolis, Minn.). The BrdU assay was performed according to the manufacturer's specifications (Roche Molecular Biochemicals, Indianapolis, Ind.) using a 4 h BrdU incorporation time.

### Results and Conclusions

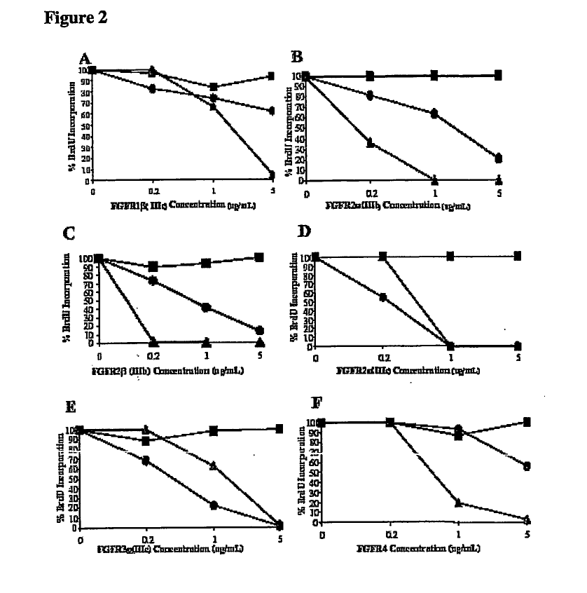

To determine the receptor binding specificity of CG53135, we examined the effect of soluble FGFRs on the induction of DNA synthesis in NIH 3T3 cells by recombinant CG531354-01 produced in E. coli. Soluble receptors for FGFR1β (IIIc), FGFR2β (IlIb), FGFR2α (IIIb), FGFR2α (IIIc), FGFR3α (IIIc), and FGFR4 were utilized. We found that soluble forms of each of these FGFRs were able to specifically inhibit the biological activity of CG53135 (FIG. 2).

Complete or nearly complete inhibition was obtained with soluble FGFR2α (IIIb), FGFR2β (IIIb), FGFR2α (IIIc), and FGFR3α (IIIc), whereas partial inhibition was achieved with soluble FGFR1β (IIIc) and FGFR4. None of the soluble receptor reagents interfered with the induction of DNA synthesis by PDGF-BB (FIG. 2), thereby demonstrating their specificity. The integrity of each soluble receptor reagent was demonstrated by showing their ability to inhibit the induction of DNA synthesis by aFGF, a factor known to interact with all of the FGFR's under analysis (FIG. 2).

### Example 5

### Protein Expression and Purification of CG53135-12 and CG53135-05

The procedure used to generate the D206N mutation in the plasmid CG53135-05/pET24a incorporated two complementary primers, containing the D206N mutation (converting a GAC->AAC), by amplifying the CG53135-05/pET24a template using Pfu, a high-fidelity polymerase. The amplified product was then digested with Dpn1 and transformed into chemically competent Top10 cells (Invitrogen). Transformants containing the desired D206N mutation were screened by sequencing the 3′-end of the CG53135-05 insert using a T7-terminator primer. The sequence integrity of the entire CG5 gene of the D206N mutant was verified using primers to the T7-promoter and T7-terminator of the pET vector.

The E. coli strain BRL (DE3) (Novagen, Madison, Wis.) was used as host for the expression of CG53135-12 protein. The cells were transformed with expression vector pET24a+ (Novagen, Madison, Wis.) carrying the coding sequence of CG53135-12. The transformed bacteria were grown either in LB broth or Terrific Broth containing 30 ug/ml kanamycin and 12.5 ug/ml tetracycline at 37° C. Expression of recombinant CG53135-12 protein was induced by adding 1 mM IPTG when the culture reached an O.D.₆₀₀ of 1.0-1.5. The induced cells were allowed to grow at 37° C. for another 3 hours and thereafter collected by centrifugation.

The harvested cell paste was resuspended in lysis buffer and subjected to high-pressure homogenization. The recombinant protein was captured by Ion Exchange chromatography (Pharmacia SP-Sepharose Fast Flow). The captured protein was eluted from column and precipitated by ammonium sulfate. The precipitate was dissolved, protein purified by Hydrophobic Interaction Chromatography (HMC) and polished by Ion Exchange chromatography (Pharmacia SP Sepharose High Performance).

E. coli BLR (DE3) cells (NovaGen) were transformed with CG5313545-05 (full-length, codon-optimized) using pET24a vector (NovaGen) and the protein purification protocol described for CG53135-12 were followed for CG53135-05 production.

### Example 6

### Confirmation of the Repalcement of Aspartic Acid 206 with Asparagine in CG53135-12

This experiment was performed to confirm the replacement of aspartic acid 206 in CG53135-05 with asparagine in CG53135-12.

The predicted average molecular weight of CG53135-05, residues 3-211, is 23296.40 Da, whereas the replacement of the aspartic acid with asparagine lowers the average molecular weight to 23295.41 Da. Accurate molecular weight determination with a quadrupole-time-of-flight mass spectrometer, capable of 5 ppm mass accuracy, can differentiate the expected 1 Da mass difference.

CG53135-12 was diluted to a final concentration of 0.8 mg/mL with the CG53135-05 formulation buffer. Five microliters of either CG53135-05 or CG53135-12 was injected onto a C4 reverse-phase HPLC column (Vydac 250×1 mm; 5 μm particle size). The variants of each protein were separated at a flow rate of 200 μl/min with a gradient consisting of 0.1% trifluoroacetic acid and acetonitrile. Eluting peaks were detected by both UV/Vis at 280 nm and by positive-ion electrospray ionization. The mass spectrometer was scanned from 500-2500 m/z. Collected data were processed with the Maximum entropy software for accurate molecular weight determination.

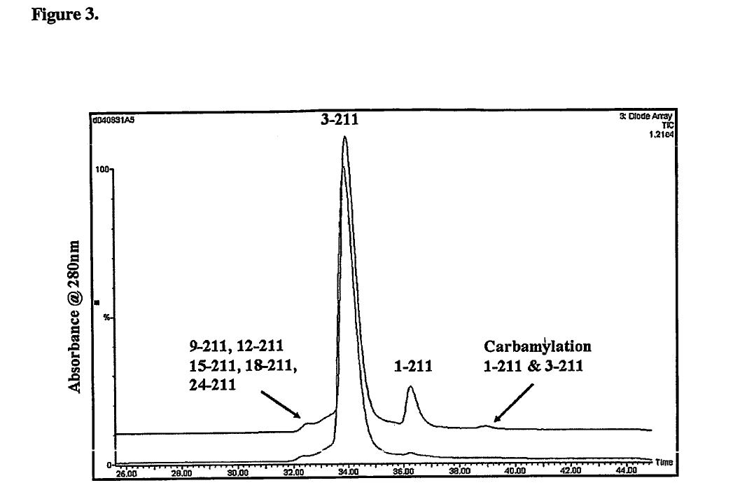

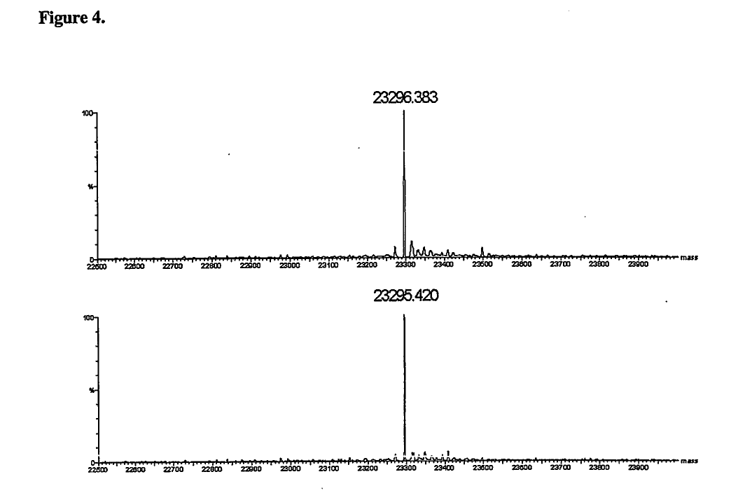

The chromatographic profiles for both CG53135-05 (FGF-20) and CG53135-12 were reasonably similar considering the two proteins were manufactured at different scales (FIG. 3). The primary difference observed was a 1 Da shift in molecular weight for all of the peaks (FIG. 4). The major peak corresponds to a C-terminal truncated variant of the sequence (residues 3-211). The expected average molecular weight for CG53135-05, residues 3-211, is 23296.397 and the observed molecular weight is 23296.383. The similar variant of CG53135-12 is expected to be 0.985 Da lower in mass or 23295.412. The observed molecular weight is 23295.420, corresponding to a difference of 0.96 Da.

In summary, the observed mass difference is consistent with the intended replacement of and aspartic acid with an asparagine to create the new molecular entity, CG53135-12.

### Example 7

### Comparison of Proliferative Activity Between CG53135-05 and CG53135-12

Proliferative activity of CG53135-05 and the SNP variant CG53135-12 were tested in various cell lines by BrdU incorporation or Cell titer blue/Alamar blue assay.

BrdU Incorporation: Proliferative activity was measured by treatment of cultured NIH-3T3 murine fibroblast cells with either conditioned media containing CG53135-12 or CG53135-05 protein, or purified CG53135-12 or CG53135-05 protein for 24 to 72 hours, followed by measurement of BrdU incorporation during DNA synthesis. Cells were incubated with BrdU (10 μM final concentration) for 3 hours and BrdU incorporation was assayed according to the manufacturer's specifications (Boehringer Mannheim, Indianapolis, Ind.).

Cell Titer-Blue/Alamar blue assay: Proliferative activity was measured by treatment of cultured Balb/MK murine keratinocytes or murine Baf3 pre-B cell line with media containing purified CG53135-12 or CG53135-05 for 24 to 72 hours. Alamar blue (Biosource International, Calif.) or Cell titer blue (Promega Corporation, Wis.) was added to the cell culture according to the manufacturer's specifications. Change in color by the indicator dye was measured at 570/630 nm. In general, the greater the change in dye color relative to untreated or control cells, the greater the proliferative and/or metabolic rate.

### Results:

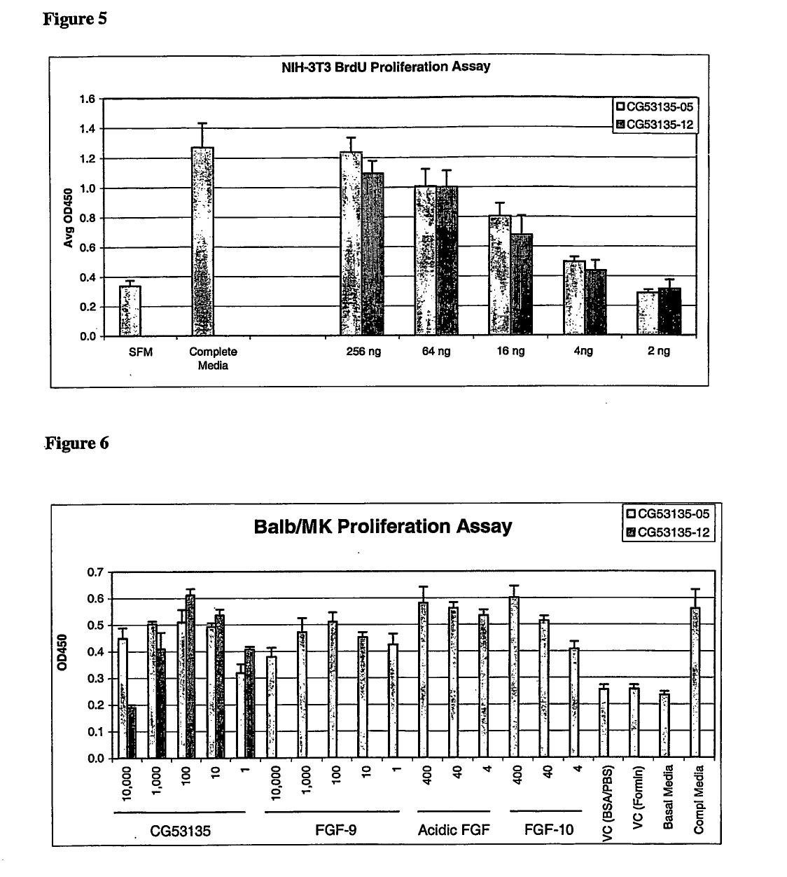

CG53135-12 and CG53135-05 induced DNA synthesis of NIH-3T3 murine fibroblasts, in a dose dependant manner between 2 ng/ml and 256 ng/ml, as measured by incorporation of bromodeoxyuridine (FIG. 5). Results at the 256 ng/ml are comparable with the complete media (containing 10% FBS) control.

CG53135-12 and CG53135-05 also induced proliferation of Balb/MK murine keratinocytes, as measured spectrophotometrically by Alamar blue color change. The activity was maximal in the 100 ng/ml range, and diminished at higher or lower concentration (FIG. 6). The results were comparable to the FGF-9 control.

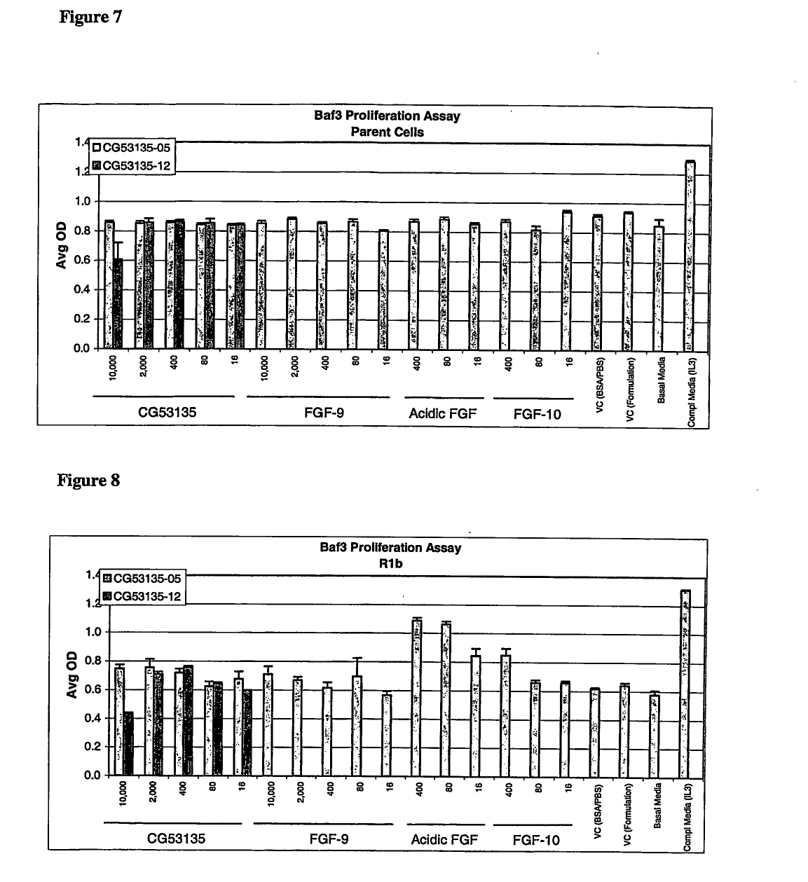

However, CG53135-12 and CG53135-05 did not induce proliferation of the parental murine Baf3 pre-B cell line, which does not express any FGF receptors. The control FGFs (FGF-9, 10 and acidic FGF) also did not induce proliferative activity (FIG. 7).

CG53135-12 and CG53135-05 and FGF-9 did not induce proliferation of the murine Baf3 pre-B cell line transfected with the mouse FGF receptor R1b (FIG. 8). FGF-10 and acidic FGF were mitogenic, as expected.

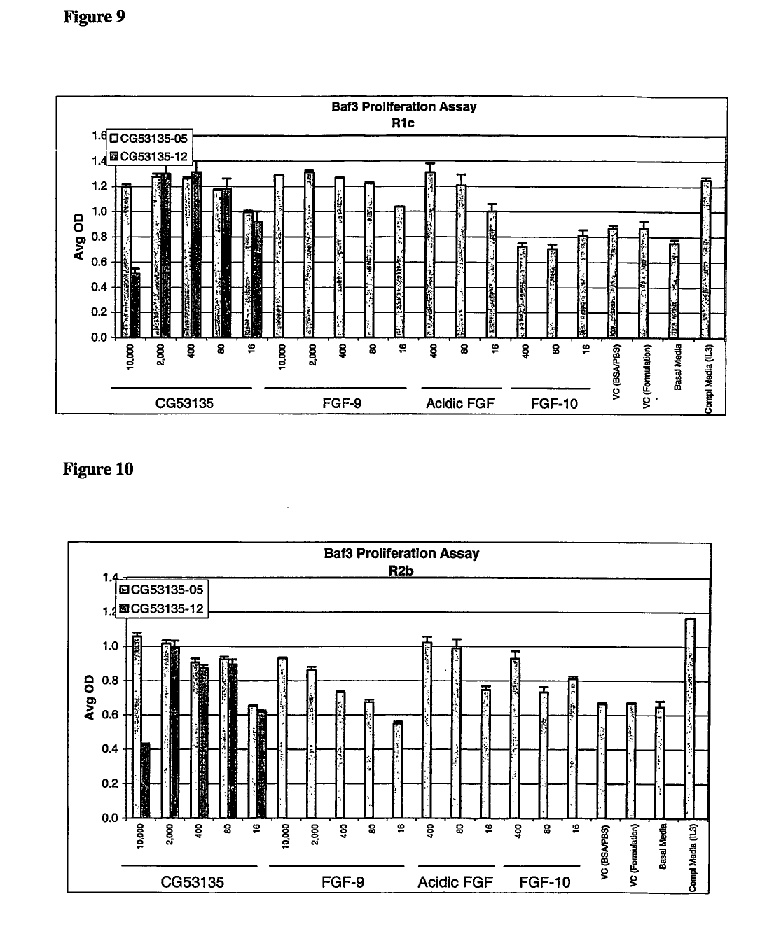

CG53135-12, CG53135405, FGF-9 and acidic FGF induced proliferation, in a dose dependant manner, of the murine Baf3 pre-B cell line transfected with the mouse FGF receptor R1c. As predicted, FGF-10 (KGF-2) did not induce proliferation of the “c-type” receptor (FIG. 9).

CG53135-12, CG53135-05, FGF-9, FGF-10 and acidic FGF induced proliferation, in a dose dependant manner, of the murine Baf3 pre-B cell line transfected with the mouse FGF receptor R2b (FIG. 10).

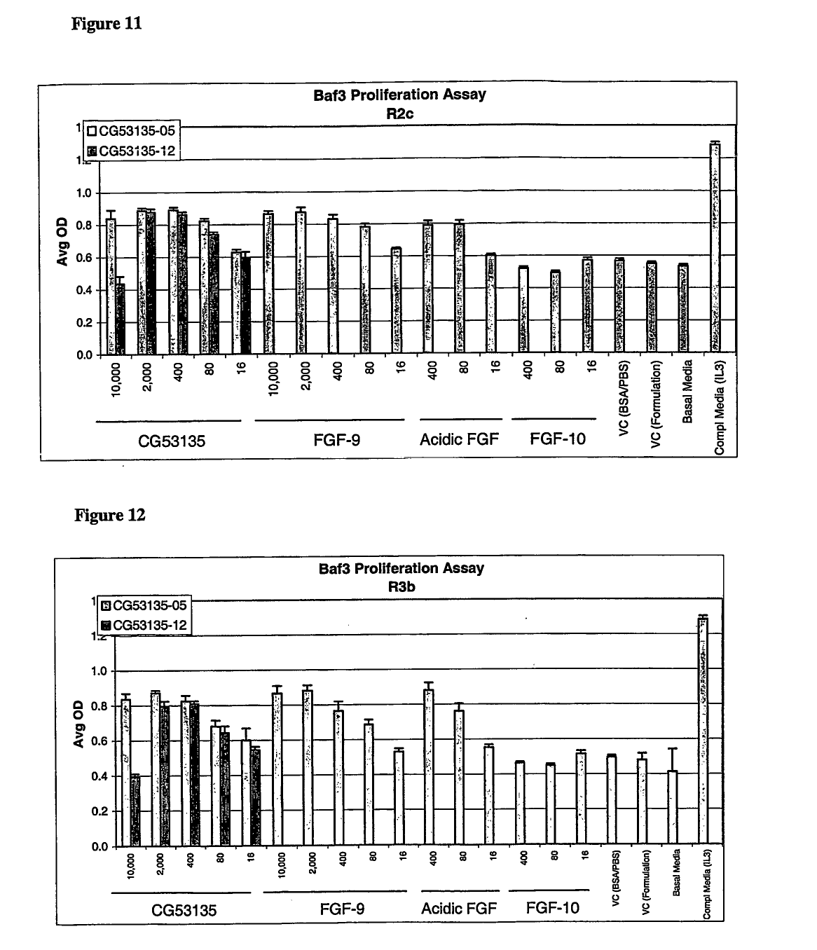

CG53135-12, CG53135-05, FGF-9 and acidic FGF induced proliferation, in a dose dependant manner, of the murine Baf3 pre-B cell line transfected with the mouse FGF receptor R2c. As predicted, FGF-10 (KGF-2) did not induce proliferation of the “c-type” receptor (FIG. 11).

CG53135-12, CG5313545, FGF-9 and acidic FGF induced proliferation, in a dose dependant manner, of the murine Baf3 pre-B cell line transfected with the mouse FGF receptor R3b. FGF-10 is not mitogenic in cells transfected with the FGF receptor 3b (FIG. 12).

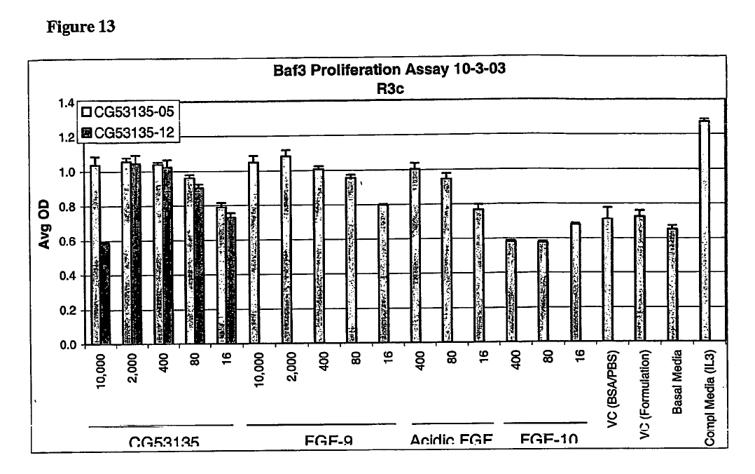

CG53135-12, CG53135-05, FGF-9 and acidic FGF induced proliferation, in a dose dependant manner, of the murine Baf3 pre-B cell line transfected with the mouse FGF receptor R3c. As predicted, FGF-10 (KGF-2) did not induce proliferation of the “c-type” receptor (FIG. 13).

Results of the proliferation assays suggest that the CG53135-12 can be used in proliferation-related disorders such as tumors, restenosis, psoriasis, diabetic and post-surgery complications, and arthritis (e.g., osteoarthritis or rheumatoid arthritis) and others. In addition, as the proliferation data are comparable to CG53135-05, indicating that CG53135-12 has functions similar to that of CG53135-05 (U.S. Ser. No. 10/435087, the disclosure of which is incorporated herein in its entirety) and thus could be used as a therapeutic for oral mucositis (U.S. Ser. No. 10/435087, the disclosure of which is incorporated in toto herein), inflammatory bowel disease (U.S. Ser. No. 09/992840, the disclosure of which is incorporated in toto herein) and stroke (U.S. Ser. No. 60/469353, the disclosure of which is incorporated in toto herein). Furthermore, proliferation and migration of neuronal progenitor cells, neuronal and glial differentiation, neurite extensions, and synapse formation has been shown in other FGF-family members, which coupled with the CG53135-12 data suggest similar functions for FGF-20. This may further support use of CG53135-12 or CG53135-05 as a therapeutic for stroke and other disorders.

Although proliferation data shows that both CG53135-05 and CG53135-12 have similar mitogenic activity, these assays are generally not able to distinguish between protein concentrations at or above 10 μg/ml, the concentration at which one would expect to see the transition from monomer to dimer beginning for FGF-20.

### Example 8

### Size Exclusion Chromatography with Light Scattering

Molecular modeling of the aspartic acid to asparagine substitution in CG53135-12 predicts that the SNP is less likely to dimerize at similar concentrations than the wild type CG53135-05 (See Example 3). This modeling is based on the published crystal structure of FGF-9 (68% homologous), and it is predicted dimerization domains (Journal of Biological Chemistry 2001, 276(6): 4322-29). To test this hypothesis, Size Exclusion Chromatography with light scattering studies were carried out using a Superdex-75 column (Pharmacia) connected in-line with ultraviolet, laser light scattering and refractive index detectors. The buffer contained 3% glycerol, 40 mM sodium acetate, and 200 mM arginine. Molecular weight was determined by solving the equation that relates the excess scattered light, measured at several angles, to the concentration of solute and the weight-average molar mass (ASTRA calculation). These values were also compared to known molecular weight standards run under the same conditions.

### Results:

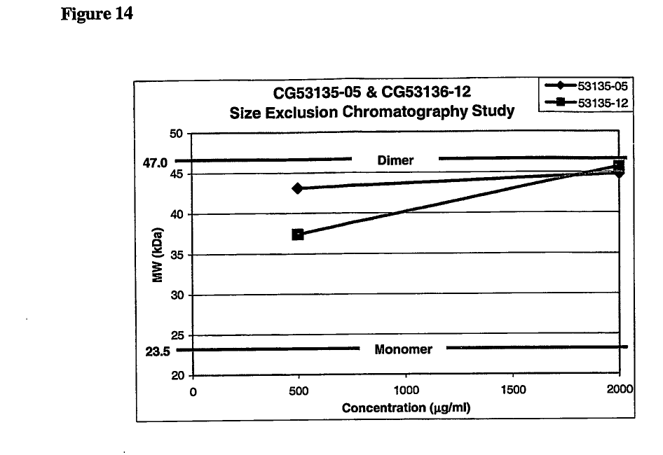

At 2 mg/ml, CG53135-12 exists exclusively in the dimeric form. At 0.5 mg/ml, CG53135-12 exists in a state that is partly monomeric and partly dimeric. This confirms the FGF-9 findings, which transitions from a monomer to dimmer in a concentration dependant manner between 8 μg/ml and 2 mg/ml (FIG. 14).

### Example 9

### Biocore Ananlysis of CG53135-12 and CG5135-05

Biacore technology is based on Surface Plasmon Resonance (SPR), and monitors bio-molecular interactions in real time. SPR is an optical phenomenon occurring at the interface of two surfaces with different refractive indexes when a light beam hits the low refractive index surface at a certain angle. The result of this SPR effect is a reduction of the light intensity that is being reflected. This change in SPR angle is expressed in Resonance Units (RUs) and is proportionate to the amount of macromolecules bound. The changes of the resonance signal (RUs) as a function of time are reported in a sensorgram.

The system contains microchips on which ligands are immobilized so analytes or buffers can be passed over to measure molecular interactions.

For the immobilized phase, CM5 BIAcore chips coated with a high binding capacity carboxymethylated dextran matrix were used in these experiments. The receptors were immobilized at a high density on the dextran surface, covalently bound by amine coupling. The surface densities were tested and were nearly equivalent at ˜5,000 RU. The 10 commercially available human FGF receptors (R&D Systems), the positive control (an anti-CG53135 monoclonal antibody) and two negative controls (human irrelevant isotype-matched antibodies) were all coated on the microchips. Each chip has 4 flow cells; flow cell #1 was used as a blank, and flow cells #2, #3 and #4 represented the receptors or control proteins. The FGF receptors consist of the extracellular domains fused to the carboxy terminal Fc region of human IgG1 with a protein linker.

For the mobile phase, we tested 6 serial dilutions of CG53135-05 and CG53135-12:3.125 nM, 6.25 nM, 12.5 nM, 25.0 nM, 50.0 nM and 100.0 nM. For both variants, 4 iterations of each concentration were run. The different concentrations of analytes were run on the chips in a random fashion.

The assay conditions used were: analyte injected at 60 ul/min, association time 3 min, dissociation time 2 min, regeneration with 1M NaCl after each binding for 30 s at 50 ul/min. The running buffer was HBS-EP (0.1 HEPES pH 7.4, 0.15M NaCl, 3 mM EDTA, and 0.005% Surfactant P20).

### Results:

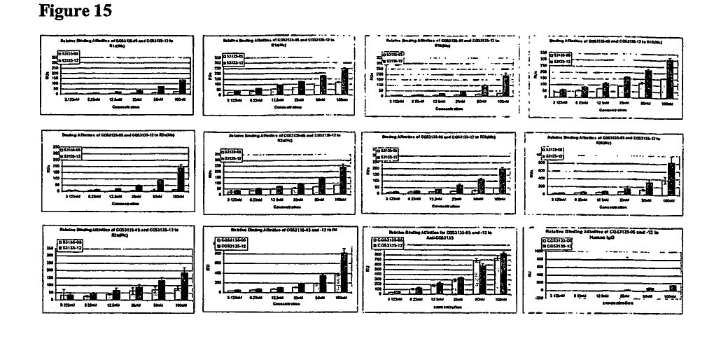

When tested between 3.1 nM and 100.0 nM, CG53135-12 displayed higher RUs against the 10 human FGF receptors than CG53135-05 at all concentrations (FIG. 15). Both proteins exhibit a dose-dependent binding pattern. Both CG53135-05 and CG53135-12 show a similar profile on the chip with the positive control, anti-CG53135 antibody, which infers that the protein concentrations of both CG53135s are normalized correctly. However, CG53135-12 did show more binding to the negative control antibody. Therefore, a trend towards a generally higher background binding for CG53135-12 would be a plausible explanation for the higher RUs of CG53135-12 over CG53135-05.

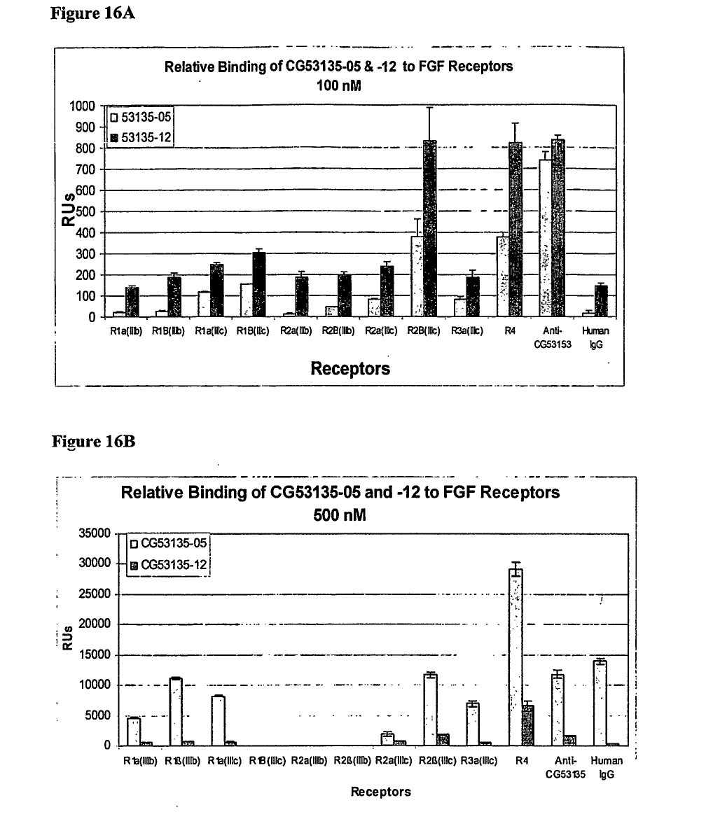

FIG. 16 shows a comparison of CG53135-05 and CG53153-12 at 100 nM, the highest concentration that can be accurately measured. Since there should be no binding to the negative control human IgG, any RUs measured for these samples can be considered background staining and should be subtracted from all the samples. When this is done, CG53135-12 displays significantly higher binding for receptors R2b(IIIc) and R4 than CG53135-05 does.

At 500 nm, CG53135-05 displayed a higher binding to all the receptors tested and also on the negative control than CG53135-12. The sensorgram pattern generated by CG53135-05 at this concentration is characteristic of non-specific protein interactions such as analyte adsorbing to the tubing, mass transport, and protein aggregation or precipitation. Therefore, no binding affinities to the FGF receptors could be determined for CG53135-05 at this concentration. This phenomenon was not observed with CG53135-12, and respective binding to each of the FGF receptors could be determined. All together, these BIAcore data indicate that CG53135-12 behaves differently in this system than CG53135-05, and suggests that CG53135-12 may have different receptor binding affinities and/or altered stability.

### Conclusions:

The FGF receptors can be divided between binders, R1a(IIIc), R1b(IIIc), R2b(IIIc) and R4, and low or non-binders, R1a(IIIb), R1b(IIIc), R2a(IIIb), R2b(IIIb), R2a(IIIc) and R3a(IIIc), for each protein.

At 100 nM, CG53135-12 displays higher RUs for receptors R2b(IIIc) and R4 than does CG53135-05 when normalized for background binding.

In the experiment performed at 500.0 nM, CG53135-05 shows very high binding values compared to CG53135-12 for all the receptors as well as the negative controls. The sensorgrams for CG53135-05 at 500 nM are characteristic of non specific binding. This phenomenon is not observed, or is much lower, for CG53135-12.

Overall, CG53135-12 does display differential binding and stability characteristics to FGF receptors than does CG53135-05 in experiments performed on a BIAcore.

### Equivalents

Those skilled in the art will recognize, or be able to ascertain using no more than routine experimentation, many equivalents to the specific embodiments of the invention described herein. Such equivalents are intended to be encompassed by the following claims.

Thus, while the preferred embodiments of the invention have been illustrated and described, it is to be understood that this invention is capable of variation and modification, and should not be limited to the precise terms set forth. The inventors desire to avail themselves of such changes and alterations which may be made for adapting the invention to various usages and conditions. Such alterations and changes may include, for example, different pharmaceutical compositions for the administration of the proteins according to the present invention to a mammal; different amounts of protein in the compositions to be administered; different times and means of administering the proteins according to the present invention; and different materials contained in the administration dose including, for example, combinations of different proteins, or combinations of the proteins according to the present invention together with other biologically active compounds for the same, similar or differing purposes than the desired utility of those proteins specifically disclosed herein. Such changes and alterations also are intended to include modifications in the amino acid sequence of the specific desired proteins described herein in which such changes alter the sequence in a manner as not to change the desired potential of the protein, but as to change solubility of the protein in the pharmaceutical composition to be administered or in the body, absorption of the protein by the body, protection of the protein for either shelf life or within the body until such time as the biological action of the protein is able to bring about the desired effect, and such similar modifications. Accordingly, such changes and alterations are properly intended to be within the full range of equivalents, and therefore within the purview of the following claims.

The invention and the manner and process of making and using it have been thus described in such full, clear, concise and exact terms so as to enable any person skilled in the art to which it pertains, or with which it is most nearly connected, to make and use the same.

1. An isolated nucleic acid molecule selected from the group consisting of:
(a) a nucleic acid molecule comprising a nucleotide sequence selected from the group consisting of SEQ ID NOs: 1, 5, 7, 9, 10, 12, 14, 16, 18, 20, 22, and 24;
(b) a nucleic acid molecule encoding a protein comprising an amino acid sequence selected from the group consisting of SEQ ID NOs: 2, 6, 8, 11, 13, 15, 17, 19, 21, 23, and 25;
(c) a fragment of an nucleic acid molecule of any of (a)-(b); and
(d) a complement of an nucleic acid molecule of any of (a)-(c).

2. The isolated nucleic acid molecule of claim 1 comprising SEQ ID NO: 1.

3. The isolated nucleic acid molecule of claim 1 comprising a nucleotide sequence encoding an amino acid sequence of SEQ ID NO: 2.

4. A vector comprising the nucleic acid molecule of claim 1.

5. The vector of claim 4, wherein said nucleic acid molecule is operably linked to an expression control sequence.

6. A prokaryotic or eukaryotic host cell containing a nucleic acid molecule of claim 1.

7. A prokaryotic or eukaryotic host cell containing the vector of claim 4.

8. A prokaryotic or eukaryotic host cell containing the vector of claim 5.

9. A method comprising culturing the host cell of claim 7 in a suitable nutrient medium.

10. The method of claim 9, wherein said host cell is E. coli.

11. The method of claim 9 further comprising isolating a protein encoded by said nucleic acid molecule from said cultured cells or said nutrient medium.

12. An isolated protein by method of claim 11.

13. An isolated protein selected from the group consisting of
(a) a protein comprising an amino acid sequence of SEQ ID NO: 2, 6, 8, 11, 13, 15, 17, 19, 21, 23, or 25; and
(b) a fragment of the protein of (a).

14. The isolated protein of claim 13 comprising an amino acid sequence of SEQ ID NO: 2.

15. An isolated protein comprising an amino acid sequence, wherein said amino acid sequence has one or more conservative amino acid substitutions relative to SEQ ID NO: 2, 6, 8, 11, 13, 15, 17, 19, 21, 23, or 25.

16. A pharmaceutical composition comprising a pharmaceutically acceptable carrier, and a protein of claim 13.

17. A method of stimulating proliferation, differentiation or migration of epithelial cells or mesenchymal cells comprising administering to a subject in need thereof an effective amount of a composition comprising an isolated protein of claim 13.

18. The method of claim 17, wherein said composition further comprising a pharmaceutically acceptable carrier.

19. The method of any of claims 17, wherein said epithelial cells or mesenchymal cells locate at the alimentary tract of said subject.

20. The method of claim 17, wherein said epithelial cells or mesenchymal cells locate at the pulmonary tract of said subject.

21. The method of claim 17, wherein said subject is a mammal.

22. The method of claim 21, wherein said mammal is a human.

23. A method for determining the presence or amount of the protein of claim 13 in a sample, the method comprising:
(a) providing said sample;
(b) introducing said sample to an antibody that binds immunospecifically to the protein; and
(c) determining the presence or amount of antibody bound to said protein, thereby allowing the determination of the presence or amount of protein in said sample.

24. A method for determining the presence of or predisposition to a disease associated with altered levels of expression of the nucleic acid molecule of claim 1 in a first mammalian subject, the method comprising:
a) measuring the level of expression of the nucleic acid in a sample from the first mammalian subject; and
b) comparing the level of expression of said nucleic acid in the sample of step (a) to the level of expression of the nucleic acid present in a control sample from a second mammalian subject known not to have or not be predisposed to, the disease;
wherein an alteration in the level of expression of the nucleic acid in the first subject as compared to the control sample indicates the presence of or predisposition to the disease.


SEQUENCE LISTING (24 sequences)

SEQ ID NO: 1 - DNA, 633 bp, Homo sapiens
  CDS (1)..(633)
atggctcccttagccgaagtcgggggctttctgggcggcctggagggcttgggccagcaggtgggttcgcatttcctgttgcctcctgccggggagcggccgccgctgctgggcgagcgcaggagcgcggcggagcggagcgcgcgcggcgggccgggggctgcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaagaacctactgatgtacact

SEQ ID NO: 2 - protein, 211 aa, Homo sapiens [sequence omitted]

SEQ ID NO: 3 - DNA, 633 bp, Homo sapiens
  CDS (1)..(633)
atggctcccttagccgaagtcgggggctttctgggcggcctggagggcttgggccagcaggtgggttcgcatttcctgttgcctcctgccggggagcggccgccgctgctgggcgagcgcaggagcgcggcggagcggagcgcgcgcggcgggccgggggctgcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacact

SEQ ID NO: 4 - protein, 211 aa, Homo sapiens
MAPLAEVGGFLGGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 5 - DNA, 477 bp, Homo sapiens
  CDS (1)..(474)
atggctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaaaacctgctgatgtacacctaa

SEQ ID NO: 6 - protein, 158 aa, Homo sapiens
MAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKNLLMYT

SEQ ID NO: 7 - DNA, 540 bp, Homo sapiens
  CDS (1)..(537)
atggctcccttagccgaagtcgggggctttctgggcggcctggagggcttgggccagccgggggcagcgcagctggcgcacctgcacggcatcctgcgccgccggcagctctattgccgcaccggcttccacctgcagatcctgcccgacggcagcgcgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaagaacctactgatgtacacttag

SEQ ID NO: 8 - protein, 179 aa, Homo sapiens
MAPLAEVGGFLGGLEGLGQPGAAQLAHLHGILRRRQLYCRTGFHLQILPDGSAQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKNLLMYT

SEQ ID NO: 9 - DNA, 636 bp, Homo sapiens
atggctccgctggctgaagttggtggtttcctgggcggtctggagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaaaacctgctgatgtacacctaa

SEQ ID NO: 10 - DNA, 540 bp, Homo sapiens
atggctcccttagccgaagtcgggggctttctgggcggcctggagggcttgggccagccgggggcagcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaagaacctactgatgtacacttag

SEQ ID NO: 11 - protein, 179 aa, Homo sapiens
MAPLAEVGGFLGGLEGLGQPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKNLLMYT

SEQ ID NO: 12 - DNA, 630 bp, Homo sapiens
  CDS (1)..(627)
ccgctggctgaagttggtggtttcctgggcggtctggagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaaaacctgctgatgtacacctaa

SEQ ID NO: 13 - protein, 209 aa, Homo sapiens
PLAEVGGFLGGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKNLLMYT

SEQ ID NO: 14 - DNA, 612 bp, Homo sapiens
  CDS (1)..(609)
ggtttcctgggcggtctggagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaaaacctgctgatgtacacctaa

SEQ ID NO: 15 - protein, 203 aa, Homo sapiens
GFLGGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKNLLMYT

SEQ ID NO: 16 - DNA, 603 bp, Homo sapiens
  CDS (1)..(600)
ggcggtctggagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaaaacctgctgatgtacacctaa

SEQ ID NO: 17 - protein, 200 aa, Homo sapiens
GGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKNLLMYT

SEQ ID NO: 18 - DNA, 594 bp, Homo sapiens
  CDS (1)..(591)
gagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaaaacctgctgatgtacacctaa

SEQ ID NO: 19 - protein, 197 aa, Homo sapiens
EGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKNLLMYT

SEQ ID NO: 20 - DNA, 567 bp, Homo sapiens
  CDS (1)..(564)
cacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaaaacctgctgatgtacacctaa

SEQ ID NO: 21 - protein, 188 aa, Homo sapiens
HFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKNLLMYT

SEQ ID NO: 22 - DNA, 447 bp, Homo sapiens
atcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaaaagttccagaattgtacaaggacctactgatgtacact

SEQ ID NO: 23 - protein, 149 aa, Homo sapiens
ILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKNLLMYT

SEQ ID NO: 24 - DNA, 537 bp, Homo sapiens
atggctcccttagccgaagtcgggggctttctgggcggcctggagggcttgggccagccgggggcagcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaagaacctactgatgtacact